(19) United States
(12) Patent Application Publication
(10) Pub. No.: US 2023/0272438 A1
(43) Pub. Date: Aug. 31, 2023

(54) COMPOSITIONS, SYSTEMS, AND METHODS FOR ARTIFICIAL CARBON FIXATION, CHEMICAL SYNTHESIS, AND/OR PRODUCTION OF USEFUL PRODUCTS

(71) Applicant: RUBI LABORATORIES, LLC, Middletown, DE (US)

(72) Inventor: Neeka Alison MASHOUF, Middletown, DE (US)

(73) Assignee: Rubi Laboratories, Inc., Middletown, DE (US)

(21) Appl. No.: 18/017,834

(22) PCT Filed: Jul. 26, 2021
(86) PCT No.: PCT/US2021/043232

§ 371 Date: Jan. 24, 2023

Related U.S. Application Data
(60) Provisional application No. 62/706,013, filed on Jul. 26, 2020.

(51) Int. Cl. C12P 7/24; C12N 9/12; C12N 9/10; C12N 9/90; C12N 9/02; C12P 19/02; C12N 9/88; C12N 9/16

(52) U.S. Cl. CPC C12P 7/24; C12N 9/1229; C12N 9/1022; C12N 9/90; C12N 9/0069; C12P 19/02; C12N 9/88; C12N 9/1205; C12N 9/16; C12N 9/0008; C12Y 102/01012; C12Y 207/04001; C12Y 202/01001; C12Y 401/01039; C12Y 207/01019; C12Y 503/01001; C12Y 301/03011; C12Y 501/03001; C12Y 503/01006; C12Y 301/03037

(57) ABSTRACT

Provided herein are production systems and methods to produce a plurality of organic carbon-containing compounds from carbon dioxide, including glyceraldehyde 3-phosphate, glucose, cellulose, and starch, using stabilized enzymes in aqueous media.

### CROSS-REFERENCE TO RELATED APPLICATIONS

This application claims priority to U.S. Provisional Patent Application No. 62/706,013, filed Jul. 26, 2020, which is incorporated herein by reference in its entirety.

### FIELD

The present disclosure relates generally to the production of organic carbon-based products, such as biological macromolecules, using carbon dioxide as a starting material. More specifically, the present disclosure relates to the production of various products, such as cellulose and starch from carbon dioxide using stabilized enzymes, that may be used in various industries, such as clothing, food and plastic manufacturing.

### BACKGROUND

Creating a resilient, sustainable global economy hinges on the development of new production methods and materials. In many ways, Earth, the global economy, and the future of living creatures are threatened by unsustainable production and products. For example, excess carbon emissions causing global warming, dwindling arable land for food and materials production, plastic and microplastic pollution from harmful packaging and clothing, and water scarcity and droughts while agriculture guzzles water resources.

These global issues frame an opportunity to generate important resources from unconventional, new, and sustainable systems. Thus, there is need in the art for systems and methods for the production of organic carbon-based products, such as biological macromolecules, using carbon dioxide as a starting material.

### BRIEF SUMMARY

In some aspects, provided herein are compositions, systems, and methods for artificial and/or synthetic production of compounds, materials, organics, and products. According to some embodiments, provided are compositions, systems, and methods of production of organic carbon-based products from carbon dioxide or carbon sources and involving carbon fixation or conversion reactions. According to some embodiments, provided are compositions, systems, and methods of chemical, physical, and/or biological production.

In one aspect, provided is a composition comprising or consisting essentially of a complex of a catalyst and compounds where the composition mitigates negative impacts of its environment on activity or longevity of the catalyst if it were not complexed in said environment. The complex of compounds and catalyst enables catalyst activity and longevity in various non-native environments.

In some embodiments, the catalyst is selected from enzymes, active proteins, artificial catalysts, or synthetic catalysts. In certain embodiments, the enzymes are in an activated state in the composition. In certain embodiments, the compounds are selected from synthetic polymers, natural polymers, monomers, polymer structures, micelles, heteropolymers polymerized from some methacrylate-based monomers (such as methyl methacrylate (MMA), oligo(ethylene glycol) methacrylate (OEGMA), 3-sulfopropyl methacrylate potassium salt (3-SPMA), 2-ethylhexyl methacrylate (2-EHMA)), metal organic frameworks, and compounds mimicking disordered proteins. In some variations, the compounds mimic naturally disordered proteins. In some variations, the catalyst is obtained from plant matter, natural sources, artificial sources, microbe fermentation, bioengineered microbes, and/or a supplier. In some variations, the catalyst may not be complexed with polymers if able to maintain activity and longevity in a non-native environment. In other variations, one or more catalyst(s) and/or composition(s) are incorporated into and/or onto a polymer structure, metal organic framework, microstructure, nanostructure, structure, substrate, cell, and/or reactor.

In some embodiments, the environment is non-native in terms of temperature, pH, pressure, or other characteristics. In some variations, the composition includes the environment and/or reaction vessel; and/or the catalyst may be selected from enzymes including but not limited to ribulose 1,5-bisphosphate carboxylase (RuBisCO), Rubisco activase, activases, cellulose synthase, starch synthase, starch branching enzyme, amylases, chitin synthase III, pyruvate carboxylase, fatty acid synthase, acetyl CoA carboxylase, Lys201, Lys334, Lys175, carboxylases, enzymes involved in synthesis of biological macromolecules, and enzymes involved in carbon fixation, enzymes produced in natural or engineered cells, enzymes produced by directed evolution.

In another aspect, provided is a method of making a disclosed composition comprising or consisting essentially of: mixing the enzyme and compounds in an aqueous solution; drying the mixture; and resuspending the dried mixture in a solution, forming the composition.

In another aspect, provided is a method of making a disclosed composition comprising or consisting essentially of mixing the enzyme and compounds in a media which allows high enzyme activity such that they form a complex; and drying the mixture, forming the composition.

In another aspect, provided is a method of making a disclosed composition comprising or consisting essentially of mixing the enzyme and compounds in a solution which allows high enzyme activity such that they form the composition.

In another aspect, provided is a method of using a disclosed composition comprising detecting activity of the catalyst in the composition.

In another aspect, provided is a composition comprising of microbe cells encapsulated in a protective layer where the composition mitigates the negative impact of the environment on the activity or longevity of the microbes if it were not encapsulated in said environment. The composition is resistant to various environments and can be stored while keeping the active microbes protected for extended periods of time.

In some embodiments, at least some part of the microbes is engineered or directionally evolved by bioengineering techniques; the microbes are selected from yeast, bacteria, eukaryotic cells, prokaryotic cells, algae, protists, fungi, plants, and viruses; the protective layer is selected from chemical compounds, polymers, and dry microbe cells; the protective layer includes growth media for the microbe; the protective layer dissolves or dissociates when using the composition in an environment that is not excessively harmful to the microbes; the composition includes ascorbic acid; and/or the composition is embedded on or in a material.

In another aspect, provided is a method of making a disclosed composition comprising or consisting essentially of mixing microbes with growth media and/or other compounds; separating; and drying the mixture.

In embodiments, the microbes are first genetically engineered or metabolically engineered using a technique including but not limited to molecular cloning, gene delivery, directed evolution, rational design, and genome editing; the microbes are genetically engineered to have an altered metabolism; the microbes are metabolically engineered to consume a carbon-based feedstock including a CO₂-based feedstock; the mixture is not fully dried; and/or the mixture is dried to form granules each with a protective-layer shell.

In another aspect, provided is a system comprising: a disclosed composition of encapsulated microbes; a solution which hydrates the encapsulated microbes; and microbe feedstock to feed the microbes, wherein the composition is able to be active and grow and produce products.

In another aspect the invention provides a composition consisting or comprising essentially of engineered microbe cells containing a disclosed composition involving a complex of catalyst and compounds such that the catalysis pathway is activated within the microbe. The combination of biological and synthetic compositions and processes enables higher throughput of desired reactions within the microbe.

In some embodiments, at least some part of the microbes is engineered or directionally evolved by bioengineering techniques; the microbes are selected from yeast, bacteria, eukaryotic cells, prokaryotic cells, algae, protists, fungi, plants, and viruses; and/or the microbe produces other necessary inputs to conduct the desired reactions.

In another aspect, provided is a method of making a disclosed composition comprising or consisting essentially of introducing a foreign complex of catalyst and compounds into a microbe cell structure such that the catalyst maintains its activity within the cell.

In another aspect, provided is a system of artificial carbon fixation and product synthesis and/or processing, comprising of: a disclosed composition involving a catalyst to catalyze a reaction in the system; a carbon source as an input; at least one reaction which is catalyzed in some way by a disclosed composition; at least one reaction in the system is a carboxylation reaction; a source capable of donating electrons for at least one reaction; at least some of the reactions are sequential and use some products from a reaction as reactants for a following reaction; and products are produced at least in part from the carbon source.

In some embodiments, various parts of the system exist together or separately in reaction vessels, reactors, cells, microbes, polymeric materials, polymeric structures, metal organic frameworks, microstructures, living organisms, biomedical devices, microfluidic devices, macrostructures, and/or nanostructures.

In some embodiments, the system exists in one or more reaction media; a disclosed composition is immobilized on or in a material in order to enable a continuous process with product removal; and reactants are from artificial, synthetic, or natural sources.

In some embodiments, the carbon source is selected from carbon dioxide (CO₂), methane, carbon monoxide, and C1 carbon molecules. In certain embodiments, the carbon dioxide comes from industrial output, energy-production output, waste products, and/or direct air capture of ambient air on a planet. In certain embodiments, the carbon dioxide input is released from a storage structure or material such as a metal organic framework; and the system is an artificial synthesis system.

In some embodiments, the system involves organic synthesis, inorganic synthesis, multistep synthesis. In certain embodiments, the system in part mimics a biological system.

In some embodiments, the electron source is selected from ATP, NADPH, electron donor molecules, electricity delivered through a substrate, or electricity delivered through the process environment, a cathode electrode, natural minerals, renewable energy.

In some embodiments, the system mimics at least partially a carbon fixation pathway such as the Calvin Cycle in plants; various parts of the system are connected to or easily accessible by external industrial facilities to enable on-site or near on-site function; and/or any of the products include carbon-containing compounds and/or polymers for example, but not limited to, monosaccharides, polysaccharides, carbohydrates, Glycerate 3-phosphate, glyceraldehyde 3-phosphate, lipids, biological macromolecules, nucleic acids, small molecules, molecules involved in natural carbon fixation cycles, and amino acids; and/or water is a byproduct.

In another aspect, the invention provides a method of using a disclosed composition to perform artificial carbon fixation comprising or consisting essentially of: contacting a disclosed composition with a carbon source under conditions wherein the disclosed composition conducts carboxylation as part of a carbon fixation or conversion process and produces at least one carbon-containing molecule (referred to here as Product 1); at least some of the produced Product 1 molecules are reduced by an electron source to produce another product molecule (referred to here as Product 2); at least some of the molecules of Product 2 are converted into monosaccharide molecules; and at least some of the monosaccharide molecules are either exported from the process or involved in at least one additional reaction step involving a disclosed composition wherein the monosaccharide molecules are synthesized into biological macromolecules.

In another aspect, the invention provides a method of using a disclosed composition to perform artificial carbon fixation comprising or consisting essentially of: contacting a disclosed composition with a carbon source under conditions wherein the disclosed composition conducts carboxylation as part of a carbon fixation or conversion process and produces at least one carbon-containing molecule (referred to here as Product 1); at least some of the produced Product 1 molecules are converted to another product (referred to here as Product 2), using energy released from ATP hydrolysis; at least some of the molecules of Product 2 are reduced by an electron source to produce another product molecule (referred to here as Product 3); at least some of the molecules of Product 3 are converted into monosaccharide molecules; and at least some of the monosaccharide molecules are either exported from the process or involved in at least one additional reaction step involving a disclosed composition wherein the monosaccharide molecules are synthesized into biological macromolecules.

In some embodiments, the carbon source is selected from carbon dioxide, methane, carbon monoxide, and C1 carbon molecules; Product 1 is 3-phosphoglyceric acid (3-PGA), Product 2 is 1,3-bisphosphoglycerate, Product 3 is glyceraldehyde 3-phosphate (G3P), and the monosaccharide is glucose; the disclosed composition involves a catalyst; an involved disclosed composition involves microbes; the energy/electron sources are selected from sources including but not limited to ATP, NADPH, electron donor molecules, electricity delivered through a substrate, electricity delivered through the process environment, a cathode electrode, natural minerals, renewable energy, and ions; the carbon dioxide is captured from an environment and introduced into the system; the carbon dioxide is ambient to the system; the biological macromolecules are selected from carbohydrates, lipids, nucleic acids, and proteins; the biological macromolecules are processed further into higher-order structures for example, but not limited to, polymer fibers, textiles, Rayon, polymer networks, polymer gels, plastics, artificial tissue, edible polymeric material, edible substances, bioplastic, cosmetic products, crystalline polymer structures, semi-crystalline polymer structures, amorphous polymers, cross-linked polymers, emulsions, emulsions, medicine, artificial building materials, biomedical materials, paper; any of the products include carbon-containing compounds and/or polymers for example but not limited to monosaccharides, polysaccharides, carbohydrates, Glycerate 3-phosphate, glyceraldehyde 3-phosphate, lipids, biological macromolecules, nucleic acids, amino acids, small molecules; the products or exported monosaccharide molecules are used as a feedstock in a microbe fermentation process; the method is conducted at least partially in microbe cells; the method at least in part includes some reaction steps, reactants, and products of a carbon fixation pathway for example but not limited to the Calvin Cycle, Reverse Krebs Cycle, Reductive acetyl CoA pathway, 3-Hydroxypropionate bicycle, gluconeogenesis.

In certain embodiments, the method mimics at least partially reactions in a biological reaction pathway; water is produced through a dehydration synthesis reaction and is then separated out; and/or the method includes additional steps of chemical reactions such as conducting enolization, carboxylation, hydration, elongation, C—C bond cleavage, and/or protonation each involving a disclosed composition.

In another aspect, provided is a method of processing a product of a carbon fixation process to create a higher-order product, material, and/or byproducts comprising or consisting essentially of: using the product as an input to one or more synthesis reactions to create a product (referred to here as Product 1); and using a product or Product 1 as an input to one or more reaction steps to create a higher-order material. In one variation, Product 1 is glucose.

In some embodiments, the input is a saccharide; a synthesis process utilizes a disclosed composition; various parts of the system exist together or separately in reaction vessels, reactors, cells, microbes, polymeric materials, polymeric structures, metal organic frameworks, microstructures, living organisms, biomedical devices, microfluidic devices, macrostructures, and/or nanostructures; the products are carbohydrates, biological macromolecules, lipids, proteins, nucleic acids, starches, peptides, hormones, chemical compounds; the higher-order materials include but are not limited to cellulose-fiber fabric, edible material, nutritious food for humans, cardboard, paper, plastics, polymer materials, wood, biomaterials, chitin, chitosan, insulin, glycogen, synthetic tissue, emulsions, cosmetics, structural building materials, building materials, packaging materials, biomedical materials, polymer structures; and/or the byproducts include water.

In another aspect, provided is a system of chemical synthesis and/or processing, comprising of: a disclosed composition involving one or more catalysts to catalyze reactions in the system; at least one reaction which is catalyzed in some way by a disclosed composition; a source capable of donating electrons to or accepting electrons from at least one reaction; and at least some of the reactions are sequential and use some products from an initial reaction as reactants for the next reaction.

In some embodiments, various parts of the system exist together or separately in reaction vessels, reactors, cells, microbes, polymeric materials, polymeric structures, metal organic frameworks, microstructures, living organisms, biomedical devices, microfluidic devices, macrostructures, and/or nanostructures; the system exists in one or more reaction media; a disclosed composition is immobilized on or in a material in order to enable a continuous process with product removal; reactants are from artificial, synthetic, or natural sources; the system is an artificial synthesis system; the system involves organic synthesis, inorganic synthesis, multistep synthesis; the system in part mimics a biological system; various parts of the system are connected to or easy accessible by external industrial facilities to enable on-site or near on-site function; the energy/electron sources are selected from ATP, NADPH, electron donor molecules, electricity delivered through a substrate, electricity delivered through the process environment, a cathode electrode, natural minerals, renewable energy; any of the products include carbon-containing compounds and/or polymers for example, but not limited to, monosaccharides, polysaccharides, carbohydrates, Glycerate 3-phosphate, glyceraldehyde 3-phosphate, lipids, biological macromolecules, nucleic acids, small molecules, and amino acids; and/or water is a byproduct.

In another aspect, provided is a method of using a disclosed composition to perform chemical synthesis and/or processing comprising or consisting essentially of: contacting a disclosed composition with one or more reactants under conditions wherein the disclosed composition conducts a desired reaction and produces at least one product molecule (referred to here as Product 1); and at least some of the produced Product 1 molecules are reduced or oxidized by an electron donor/acceptor to produce another product molecule (referred to here as Product 2).

In another aspect, provided is a method of using a disclosed composition to perform chemical synthesis and/or processing comprising or consisting essentially of: contacting a disclosed composition with one or more reactants under conditions wherein the disclosed composition conducts a series of reactions to produce a desired product molecule.

In some embodiments, the disclosed composition involves one or more catalysts; an involved disclosed composition involves microbes; the reaction is selected from chemical reactions including but not limited to polymerization, dehydration synthesis, breakdown, synthesis, decomposition; the energy/electron sources are selected from sources including but not limited to ATP, NADPH, electron donor molecules, electricity delivered through a substrate, electricity delivered through the process environment, electrodes, natural minerals, renewable energy, and ions; the biological macromolecules are selected from carbohydrates, lipids, nucleic acids, and proteins; the biological macromolecules are processed further into higher-order structures for example but not limited to polymer fibers, textiles, Rayon, polymer networks, polymer gels, artificial tissue, edible polymeric material, edible substances, bioplastic, cosmetic products, crystalline polymer structures, semi-crystalline polymer structures, amorphous polymers, cross-linked polymers, emulsions, emulsions, medicine, artificial building materials, biomedical materials, paper; any of the products include carbon-containing compounds and/or polymers for example but not limited to monosaccharides, polysaccharides, carbohydrates, Glycerate 3-phosphate, glyceraldehyde 3-phosphate, lipids, biological macromolecules, nucleic acids, amino acids, small molecules; the products or exported monosaccharide molecules are used as a feedstock in a microbe fermentation process; the method is conducted at least partially in microbe cells; the method mimics at least partially reactions in a biological reaction pathway; water is produced through a dehydration synthesis reaction and is then separated out; and/or the method includes additional steps of chemical reactions conducting enolization, carboxylation, hydration, elongation, C—C bond cleavage, and/or protonation each involving a disclosed composition.

In another aspect, provided is a method of processing a product of a disclosed system, disclosed method, or chemical compound to create a higher-order product, material, and/or byproducts comprising or consisting essentially of: using the product as an input to one or more chemical reactions to create a product (referred to here as Product 1); and using a product or Product 1 as an input to one or more reaction steps to create a material.

In some embodiments, the input is a saccharide; a synthesis process utilizes a disclosed composition; various parts of the system exist together or separately in reaction vessels, reactors, cells, microbes, polymeric materials, polymeric structures, metal organic frameworks, microstructures, living organisms, biomedical devices, microfluidic devices, macrostructures, and/or nanostructures; the higher-order products are carbohydrates, biological macromolecules, lipids, proteins, nucleic acids, starches, peptides, hormones, the reaction steps may include chemical changes, physical changes, polymer processing, polymer engineering, synthesis reactions, decomposition reactions, chemical reactions; the higher-order materials include but are not limited to cellulose-fiber fabric, edible material, nutritious food for humans, cardboard, paper, plastics, polymer materials, wood, biomaterials, chitin, chitosan, insulin, glycogen, synthetic tissue, emulsions, cosmetics, structural building materials, building materials, packaging materials, biomedical materials, polymer structures; and/or the byproducts include water.

In some aspects, provided is a system for producing cellulose from carbon dioxide and intermediates described herein using stabilized enzymes.

In some aspects, provided is a system for producing glyceraldehyde 3-phosphate from carbon dioxide using stabilized enzymes. In some embodiments, the production system regenerates RuBP and ATP and produces glyceraldehyde 3-phosphate from carbon dioxide via the Calvin Cycle. In other embodiments, the production system regenerates RuBP and produces glyceraldehyde 3-phosphate from carbon dioxide via the Calvin Cycle using an ATP source. In some embodiments, the production system regenerates ATP and produces glyceraldehyde 3-phosphate from carbon dioxide via the Calvin Cycle using a RuBP source.

In one aspect, provided is a production system for producing glyceraldehyde 3-phosphate from carbon dioxide, comprising: a carbon dioxide source configured to output carbon dioxide; a phosphate source configured to output a phosphate agent; and a reactor configured to: receive carbon dioxide from the carbon dioxide source and the phosphate agent from the phosphate source into the reactor containing ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media to: produce glyceraldehyde 3-phosphate, regenerate ribulose 1,5-bisphosphate, and regenerate adenosine triphosphate.

In one aspect, provided is a production system for producing glyceraldehyde 3-phosphate from carbon dioxide, comprising: a carbon dioxide source configured to output carbon dioxide; a phosphate source configured to output a phosphate agent; a ribulose 1,5-bisphosphate configured to output ribulose 1,5-bisphosphate; and a reactor configured to: receive carbon dioxide from the carbon dioxide source, the phosphate agent from the phosphate source, and ribulose 1,5-bisphosphate from the ribulose 1,5-bisphosphate source into the reactor containing stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, and an electron donating source in an aqueous media to: produce glyceraldehyde 3-phosphate, and regenerate adenosine triphosphate.

In one aspect, provided is a production system for producing glyceraldehyde 3-phosphate from carbon dioxide, comprising a carbon dioxide source configured to output carbon dioxide; an adenosine triphosphate source configured to output adenosine triphosphate; and a reactor configured to: receive carbon dioxide from the carbon dioxide source and adenosine triphosphate from the adenosine triphosphate source into the reactor containing ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media to: produce glyceraldehyde 3-phosphate, and regenerate ribulose 1,5-bisphosphate.

In some aspects, provided is a system for producing glucose from carbon dioxide using stabilized enzymes. In some embodiments, the production system regenerates RuBP and ATP and produces glucose from carbon dioxide via the Calvin Cycle and gluconeogenesis pathway. In other embodiments, the production system regenerates RuBP and produces glucose from carbon dioxide via the Calvin Cycle and gluconeogenesis pathway using an ATP source. In some embodiments, the production system regenerates ATP and produces glucose from carbon dioxide via the Calvin Cycle and gluconeogenesis pathway using a RuBP source.

In one aspect, provided is a production system for producing glucose from carbon dioxide, comprising: a carbon dioxide source configured to output carbon dioxide; a phosphate source configured to output a phosphate agent; a water source configured to output water; and a reactor configured to: receive carbon dioxide from the carbon dioxide source, the phosphate agent from the phosphate source, and water from the water source into the reactor containing ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating agent in an aqueous media to: produce glyceraldehyde 3-phosphate, regenerate ribulose 1,5-bisphosphate, regenerate adenosine triphosphate, and convert glyceraldehyde 3-phosphate to glucose.

In one aspect, provided is a production system for producing glucose from carbon dioxide, comprising: a carbon dioxide source configured to output carbon dioxide; a ribulose 1,5-bisphosphate configured to output ribulose 1,5-bisphosphate; a phosphate source configured to output a phosphate agent; a water source configured to output water; and a reactor configured to: receive carbon dioxide from the carbon dioxide source, the phosphate agent from the phosphate source, and water from the water source into the reactor containing stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media to: produce glyceraldehyde 3-phosphate, regenerate adenosine triphosphate, and convert glyceraldehyde 3-phosphate to glucose.

In one aspect, provided is a production system for producing glucose from carbon dioxide, comprising: a carbon dioxide source configured to output carbon dioxide; an adenosine triphosphate source configured to output adenosine triphosphate; a water source configured to output water; and a reactor configured to: receive carbon dioxide from the carbon dioxide source, adenosine triphosphate from the adenosine triphosphate source, and water from the water source into the reactor containing ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media to: produce glyceraldehyde 3-phosphate, regenerate ribulose 1,5-bisphosphate, and convert glyceraldehyde 3-phosphate to glucose.

In other embodiments, the production system produces glucose from glyceraldehyde 3-phosphate via the gluconeogenesis pathway.

In one aspect, provided is a production system for producing glucose from glyceraldehyde 3-phosphate using stabilized enzymes, comprising: a glyceraldehyde 3-phosphate source configured to output glyceraldehyde 3-phosphate; a water source configured to output water; and a reactor configured to: receive glyceraldehyde 3-phosphate from the glyceraldehyde 3-phosphate source and water from the water source into the reactor containing, stabilized aldolase, stabilized fructose 1,6-bisphosphatase, stabilized phosphoglucose isomerase, and stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase in an aqueous media to produce glucose.

In one aspects, provided is a system for producing cellulose from carbon dioxide using stabilized enzymes. In some embodiments, the production system regenerates RuBP and ATP and produces cellulose from carbon dioxide via the Calvin Cycle, gluconeogenesis pathway, and various stabilized enzymes required for the synthesis of cellulose as described herein. In other embodiments, the production system regenerates RuBP and produces cellulose from carbon dioxide via the Calvin Cycle, gluconeogenesis pathway, and various stabilized enzymes required for the synthesis of cellulose as described herein using an ATP source. In some embodiments, the production system regenerates ATP and produces cellulose from carbon dioxide via the Calvin, gluconeogenesis pathway, and various stabilized enzymes required for the synthesis of cellulose as described herein using a RuBP source.

In one aspect, provided is a production system for producing cellulose from carbon dioxide using stabilized enzymes, comprising: a carbon dioxide source configured to output carbon dioxide; a phosphate source configured to output a phosphate agent; a water source configured to output water; and a reactor configured to: receive carbon dioxide from the carbon dioxide source, the phosphate agent from the phosphate source, and water from the water source into the reactor containing ribulose-1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media to: produce glyceraldehyde 3-phosphate, regenerate ribulose 1,5-bisphosphate, regenerate adenosine triphosphate, convert glyceraldehyde 3-phosphate to glucose, convert glucose to cellulose, and regenerate uridine triphosphate.

In one aspect, provided is a production system for producing cellulose from carbon dioxide using stabilized enzymes, comprising: a carbon dioxide source configured to output carbon dioxide; a ribulose 1,5-bisphosphate configured to output ribulose 1,5-bisphosphate; a phosphate source configured to output a phosphate agent; a water source configured to output water; and a reactor configured to: receive carbon dioxide from the carbon dioxide source, ribulose 1,5-bisphosphate from the ribulose 1,5-bisphosphate source, the phosphate agent from the phosphate source, and water from the water source into the reactor containing stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media to: produce glyceraldehyde 3-phosphate, regenerate adenosine triphosphate, convert glyceraldehyde 3-phosphate to glucose, convert glucose to cellulose, and regenerate uridine triphosphate.

In one aspect, provided is a production system for producing cellulose from carbon dioxide using stabilized enzymes, comprising: a carbon dioxide source configured to output carbon dioxide; a phosphate source configured to output a phosphate agent; an adenosine triphosphate source configured to output adenosine triphosphate; a water source configured to output water; and a reactor configured to: receive carbon dioxide from the carbon dioxide source, the phosphate agent from the phosphate source, adenosine triphosphate from the adenosine triphosphate source, and water from the water source into the reactor containing ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media to: produce glyceraldehyde 3-phosphate, regenerate ribulose 1,5-bisphosphate, convert glyceraldehyde 3-phosphate to glucose, convert glucose to cellulose, and regenerate uridine triphosphate.

In other embodiments, the production system produces cellulose from glyceraldehyde 3-phosphate via the gluconeogenesis pathway and various stabilized enzymes required for the synthesis of cellulose as described herein.

In one aspect, provided is a productions system for producing cellulose from glyceraldehyde 3-phosphate using stabilized enzymes, comprising: a glyceraldehyde 3-phosphate source configured to output glyceraldehyde 3-phosphate; a phosphate source configured to output a phosphate agent; a water source configured to output water; and a reactor configured to: receive glyceraldehyde 3-phosphate from the glyceraldehyde 3-phosphate source, the phosphate agent from the phosphate source, and water from the water source into the reactor containing, adenosine triphosphate, a stabilized adenosine triphosphate regenerating enzyme, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, stabilized phosphoglucose isomerase, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media to: convert glyceraldehyde 3-phosphate to glucose, regenerate adenosine triphosphate, convert glucose to cellulose, and regenerate uridine triphosphate.

In yet another embodiment, the production system produces cellulose from glucose via various stabilized enzymes required for the synthesis of cellulose as described herein.

In one aspect, provided is a production system for producing cellulose from glucose using stabilized enzymes, comprising: a glucose source configured to output glucose; a phosphate source configured to output a phosphate agent; and a reactor configured to: receive glucose from the glucose source and the phosphate agent from the phosphate source into the reactor containing adenosine triphosphate, a stabilized adenosine triphosphate regenerating enzyme, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media to: convert glucose to cellulose, regenerate adenosine triphosphate, and regenerate uridine triphosphate.

In other aspects, provided are methods for producing biological macromolecules and intermediates thereof from carbon dioxide using stabilized enzymes and the production systems described herein.

In one aspect, provided is a method for producing glyceraldehyde 3-phosphate from carbon dioxide using stabilized enzymes, comprising: combining carbon dioxide, a phosphate agent, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media; producing glyceraldehyde 3-phosphate; and regenerating ribulose 1,5-bisphosphate and adenosine triphosphate

In one aspect, provided is a method for producing glyceraldehyde 3-phosphate from carbon dioxide using stabilized enzymes, comprising: combining carbon dioxide, a phosphate agent, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, and an electron donating source in an aqueous media; producing glyceraldehyde 3-phosphate; and regenerating adenosine triphosphate.

In one aspect, provided is a method for producing glyceraldehyde 3-phosphate from carbon dioxide using stabilized enzymes, comprising: combining carbon dioxide, adenosine triphosphate, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media; producing glyceraldehyde 3-phosphate; and regenerating ribulose 1,5-bisphosphate.

In one aspect, provided is a method for producing glucose from carbon dioxide using stabilized enzymes, comprising: combining carbon dioxide, a phosphate agent, water, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating agent in an aqueous media; producing glyceraldehyde 3-phosphate; regenerating ribulose 1,5-bisphosphate and adenosine triphosphate; and converting glyceraldehyde 3-phosphate to glucose.

In one aspect, provided is a method for producing glucose from carbon dioxide using stabilized enzymes, comprising: combining carbon dioxide, ribulose 1,5-bisphosphate, a phosphate agent, water, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media; producing glyceraldehyde 3-phosphate; regenerating adenosine triphosphate; and converting glyceraldehyde 3-phosphate to glucose.

In another aspect, provided is a method for producing glucose from carbon dioxide using stabilized enzymes, comprising: combining carbon dioxide, adenosine triphosphate, water, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media; producing glyceraldehyde 3-phosphate; regenerating ribulose 1,5-bisphosphate; and converting glyceraldehyde 3-phosphate to glucose.

In another aspect, provided is a method for producing glucose from glyceraldehyde 3-phosphate using stabilizing enzymes, comprising: combining glyceraldehyde 3-phosphate, water, stabilized aldolase, stabilized fructose 1,6-bisphosphatase, stabilized phosphoglucose isomerase, and stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase in an aqueous media; and producing glucose.

In another aspect, provided is a method for producing cellulose from carbon dioxide using stabilized enzymes, comprising: combining carbon dioxide, a phosphate agent, water, ribulose-1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media; producing glyceraldehyde 3-phosphate; regenerating ribulose 1,5-bisphosphate and adenosine triphosphate, and uridine triphosphate; and converting glyceraldehyde 3-phosphate to glucose and glucose to cellulose.

In one aspect, provided is a method for producing cellulose from carbon dioxide using stabilized enzymes, comprising: combining carbon dioxide, ribulose 1,5-bisphosphate, a phosphate agent, water, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media; producing glyceraldehyde 3-phosphate; regenerating adenosine triphosphate and uridine triphosphate; and converting glyceraldehyde 3-phosphate to glucose and glucose to cellulose.

In one aspect, provided is a method for producing cellulose from carbon dioxide using stabilized enzymes, comprising: combining carbon dioxide, a phosphate reagent, water, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media; producing glyceraldehyde 3-phosphate; regenerating ribulose 1,5-bisphosphate and uridine triphosphate; and converting glyceraldehyde 3-phosphate to glucose and glucose to cellulose.

In one aspect, provided is a method for producing cellulose from glyceraldehyde 3-phosphate using stabilizing enzymes, comprising: combining glyceraldehyde 3-phosphate, a phosphate agent, water, adenosine triphosphate, a stabilized adenosine triphosphate regenerating enzyme, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, stabilized phosphoglucose isomerase, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media; converting glyceraldehyde 3-phosphate to glucose; regenerating adenosine triphosphate and uridine triphosphate; and converting glucose to cellulose.

In another aspect, provided is a method for producing cellulose from glucose using stabilizing enzymes, comprising: combining glucose, a phosphate agent, adenosine triphosphate, a stabilized adenosine triphosphate regenerating enzyme, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media; converting glucose to cellulose; and regenerating adenosine triphosphate and uridine triphosphate.

In some variations of the foregoing integrated systems and methods, the stabilized adenosine triphosphate regenerating enzyme comprises a kinase. In other variations, the stabilized adenosine triphosphate regenerating enzyme comprises a polyphosphate kinase. In some variations of the foregoing integrated systems and methods, the stabilized uridine triphosphate regenerating enzyme comprises a kinase. In other variations, the stabilized uridine triphosphate regenerating enzyme comprises a polyphosphate kinase. In some embodiments of the foregoing integrated systems and methods, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments of the foregoing integrated systems and methods, the method for producing cellulose from carbon dioxide using stabilized enzymes further comprises recycling the phosphate agent.

The invention encompasses all combinations of the particular embodiments recited herein, as if each combination had been laboriously recited.

### DESCRIPTION OF THE FIGURES

It is understood that the examples and embodiments described herein are for illustrative purposes only and that various modifications or changes in light thereof will be suggested to persons skilled in the art and are to be included within the spirit and purview of this application and scope of the disclosure.

The present application can be understood by reference to the following description taken in conjunction with the accompanying figures. Illustrations of some examples of some embodiments disclosed are included in the attached diagrams document.

FIG. 1 depicts an exemplary process of exemplary compositions, systems, and methods, including chemical processing through encapsulated enzymes, engineered microbes, and/or other disclosures.

FIG. 2 depicts an exemplary process of exemplary compositions, systems, and methods, including chemical synthesis through encapsulated enzymes in multistep synthesis reactions, polymer processing, and fiber processing at an industrial scale from carbon dioxide input.

FIG. 3 depicts an exemplary process including embodiments of disclosed engineered microbes. This figure includes chemical synthesis in microbes, polymer processing, and fiber processing at an industrial scale from carbon dioxide input.

FIG. 4 depicts exemplary industrial uses of some disclosed compositions, systems, and methods.

FIG. 5 depicts exemplary heteropolymers compositions and uses thereof of some embodiments of some disclosed compositions and systems.

FIG. 6 depicts an exemplary system for producing glyceraldehyde 3-phosphate from carbon dioxide using stabilized enzymes via the regenerative Calvin Cycle.

FIG. 7 depicts an exemplary system for producing glucose from glyceraldehyde 3-phosphate using stabilized enzymes via the gluconeogenesis pathway.

FIG. 8 depicts an exemplary system for producing cellulose from glucose using stabilized enzymes.

### DETAILED DESCRIPTION

The following description sets forth exemplary systems, methods, parameters and the like. It should be recognized, however, that such description is not intended as a limitation on the scope of the present disclosure but is instead provided as a description of exemplary embodiments. It is understood that the examples and embodiments described herein are for illustrative purposes only and that various modifications or changes in light thereof will be suggested to persons skilled in the art and are to be included within the spirit and purview of this application and scope of the disclosure.

Unless contraindicated or noted otherwise, in these descriptions and throughout this specification, the terms “a” and “an” mean one or more, the term “or” means and/or.

While enzymes are unmatched by synthetic catalysts in many aspects such as specificity, energy efficiency, and application in biological processes, there has been little success in stabilizing them outside of their native environment without compromising characteristics like activity or longevity. A tuned complex of heteropolymers around the enzyme may provide a balance of stability and accessibility to maintain protein folding and preserve activity and longevity of enzymes in non-natural environments.

While carbon emissions grow globally each year and threaten the future of the planet, there has been little progress in developing scalable methods to utilize carbon dioxide. Inspired by Earth's most important process, carbon fixation in plants, implementing carbon fixation and further processing industrially may be a solution to utilizing carbon dioxide in useful products.

### Production System Using Stabilized Enzymes

In some aspects, provided are production systems for producing biological macromolecules and intermediates thereof from carbon dioxide using stabilized enzymes.

For example, in some variations, ribulose 1,5-bisphosphate carboxylase-oxygenase (RuBisCO) enzyme is suspended in an aqueous solution similar to its native environment or an environment in which it is active. Carbon dioxide (CO2), captured from an industrial plant waste stream, is bubbled through the solution. The system mimics the carbon-fixing Calvin cycle, with the encapsulated (e.g., stabilized) enzyme catalyzing fixation of CO2 in part through carboxylation. For example, in some embodiments, through this process, system, and composition; 3-PGA is produced from the CO2 then synthesized into G3P which then is converted into glucose. From glucose, several processing pathways are possible. For example, in some embodiments, an encapsulated (e.g., stabilized) cellulose synthase enzyme, created by a similar encapsulation method, is introduced to the glucose with other inputs and reactants.

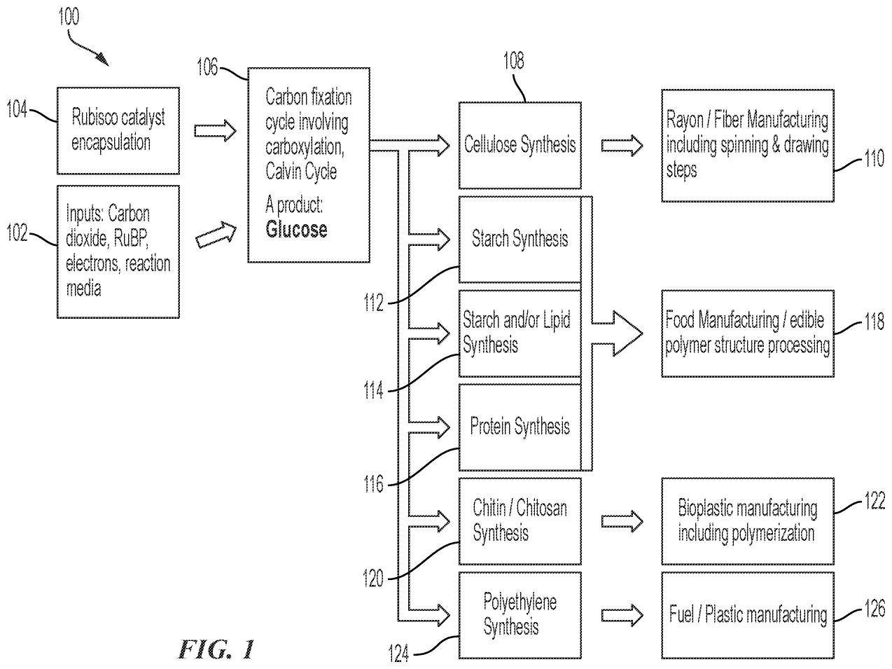

With reference to FIG. 1, exemplary process 100 depicts the production of biological macromolecules and intermediates described herein from carbon dioxide. In some embodiments, various inputs 102, including carbon dioxide, can be fed into a reactor containing a stabilized RuBisCO 102 (e.g., RuBisCO catalyst encapsulation) to produce glucose by encapsulating RuBisCO 104 with other key stabilized enzymes to produce glucose via the Calvin Cycle 106, which produces G3P, and the gluconeogenesis pathway, which converts G3P to glucose. In some embodiments, the process described herein uses one or more stabilized enzymes as described herein, e.g., for the Calvin Cycle in combination with other pathway enzymes as described herein.

With reference again to FIG. 1, the process 100 may include converting the glucose produced via the Calvin Cycle 106 and gluconeogenesis pathway to biological macromolecules (e.g., cellulose via cellulose synthesis 108, starch via starch synthesis 112, lipids via lipid synthesis 114, proteins via amino acid and protein synthesis 116, chitin via chitin synthesis 120, and/or polyethylene via polyethylene synthesis 124) using stabilized enzymes. In some embodiments, cellulose is produced from glucose using various stabilized enzymes required for the synthesis of cellulose (e.g., starch synthase) as described herein. In some embodiments, the produced cellulose is separated and extruded through a spinneret and into an acid bath to form fibers, then is processed through a similar process as a regular Rayon/natural fiber manufacturing process 110 to produce artificial natural textiles and garments or items. In some embodiments, the produced cellulose is used in a paper and cardboard production process to form paper or cardboard and packaging.

In some variations, the produced glucose is involved in further reactions or introduced to various encapsulated (e.g., stabilized) enzymes which catalyze reactions (enzymes such as starch synthase or fatty acid synthase) to produce starches, lipids, and proteins. In some embodiments, the further reactions are dehydration synthesis reactions, carbohydrate synthesis, or glycogenesis among many other possibilities. In some embodiments, the glucose may also be used as a feedstock for microbes or a disclosed composition of natural or engineered microbes which then produce a desired product, such as a biological macromolecule. In some embodiments, the starches, lipids, and proteins may be combined in various ratios and processed (such as through polymer crosslinking) to form polymer networks or gels. In certain embodiments, other compounds or nutrients may also be embedded or introduced. These produced structures may provide an edible, nutritious food source or medicine 118.

In some embodiments, glucose is processed through several steps including one involving encapsulated (e.g., stabilized) chitin synthase III and artificial chitin is produced. With reference again to FIG. 1, chitin and cellulose produced through this method are used to form a composite material which acts as a bioplastic material 122 with similar or improved properties as some plastic materials. In some embodiments, the production system produces fuel/plastic 126 using polyethylene produced from the synthesis of polyethylene 124 via stabilized enzymes as described herein.

In some embodiments, CO₂ or glucose is processed through several steps involving a disclosed composition, method, or system to produce insulin. In another variation, the system exists in a biomedical device and implanted into the body, such that it conducts a beneficial function such as insulin production and regulation.

In another variation, disclosed compositions and systems are integrated into a material, fabric, polymer structure, or another structure, and are able to conduct disclosed methods in an environment. In some variations, disclosed compositions, systems, and methods are leveraged in a desalination process. In other variations, a disclosed composition or system is fixed in or to a device and fitted to an automobile or transportation machine which typically emits carbon dioxide. The system captures and converts some carbon dioxide into another compound before it can be emitted. In another example, a disclosed composition is fixed in or onto a material which is exposed to a source of fluid (liquid or gaseous) carbon dioxide, such as ambient air, and is able to convert CO₂ into another compound.

In some embodiments of the foregoing systems and methods, the reactor is configured to receive one or more enzyme co-factors. In some variations, such enzyme cofactors may include metal ions, such as magnesium.

### Synthesis of Cellulose Using Stabilized Enzymes

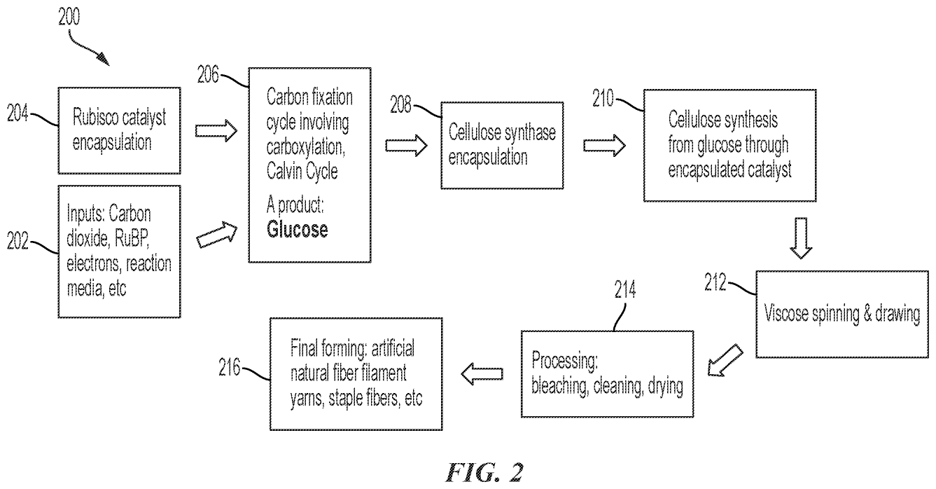

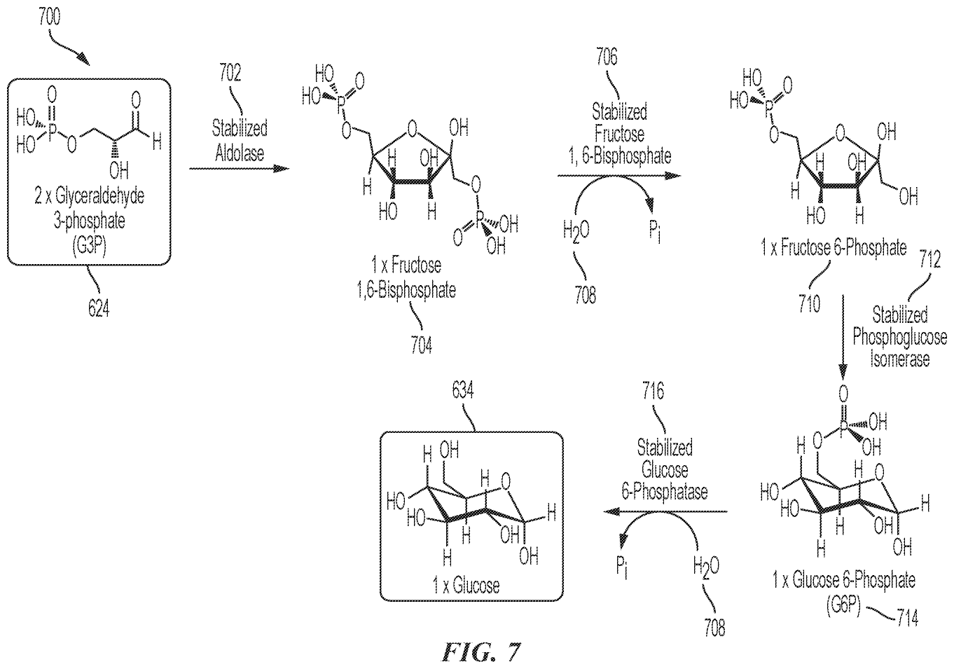

With reference to FIG. 2, exemplary process 200 depicts the production of cellulose using stabilized enzymes via a synthesis, polymer processing of cellulose to form fibers, and fiber processing at an industrial scale from carbon dioxide. In some embodiments, various inputs 202, including carbon dioxide, can be fed into a reactor containing various stabilized enzymes as described herein, including stabilized RuBisCO (e.g., RuBisCO catalyst encapsulation) 204 to produce glucose via the Calvin Cycle 206 and the gluconeogenesis pathway (e.g., FIG. 7). In some embodiments, the production systems herein uses one or more stabilized enzymes, as described herein, e.g., for the Calvin Cycle and other pathway enzymes as described herein. In some embodiments, various stabilized enzymes required for the synthesis of cellulose (e.g., cellulose synthase 208) are added to the reactor containing the glucose produced via the Calvin Cycle 206 and gluconeogenesis pathway to produce cellulose from glucose 210. In some variations of the foregoing, all required enzymes are added to the reactor at the same time. In some embodiments, the required enzymes for the synthesis of cellulose are added to the reactor to produce cellulose from glucose 210. In certain embodiments, the cellulose is further processed (e.g., viscose spinning and drawing 212 and bleaching, cleaning, and drying 214) to produce artificial fiber filament yarns and staple fibers 216.

### Synthesis of Cellulose Using Engineered Microbes

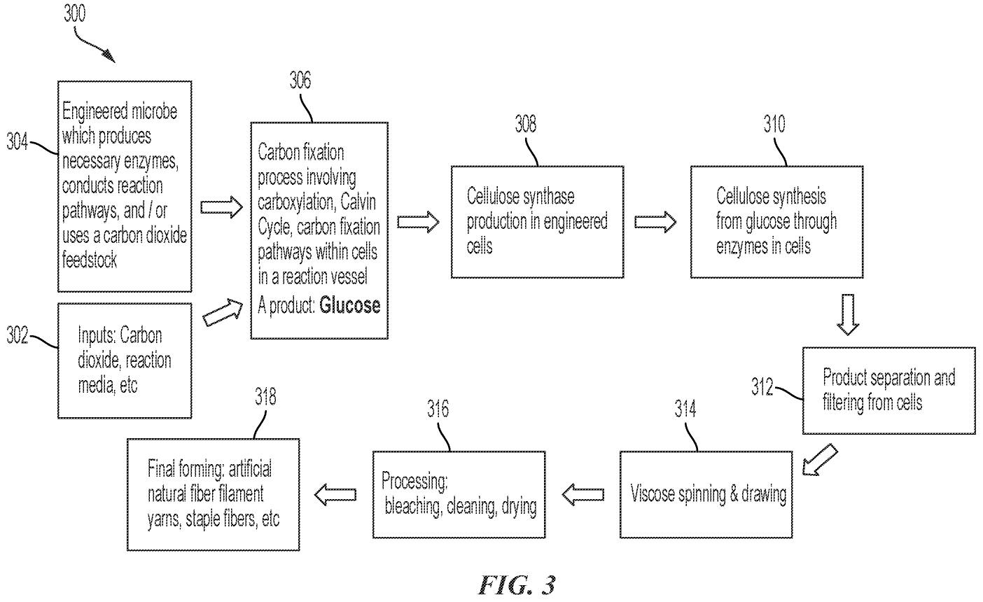

With reference to FIG. 3, exemplary process 300 depicts the production of cellulose using microbes that are metabolically engineered 304 to produce carbon fixation and synthesis enzymes and to uptake carbon dioxide 302, which enables them to fix carbon dioxide and synthesize it into desired products. In some embodiments, the glucose produced through carbon fixation 306 is used as a feedstock for microbes which consume glucose and produce other desired compounds such as biological macromolecules. For example, metabolically engineered Saccharomyces cerevisiae are fed glucose produced from carbon fixation in a reactor and produce a protein. In another embodiment, a microbe is genetically engineered to include genes to support a desired process. The microbes are introduced to encapsulated (e.g., stabilized) enzymes, in one example encapsulated (e.g., stabilized) cellulose synthase 308. The microbes then uptake glucose as feedstock, and have an always-on production pathway of creating cellulose from glucose 310 through the various possible modifications described herein. The microbes then produce the product at a high throughput. The cellulose produced from the engineered microbes may undergo further processing steps, such as product separation and filtering from cells 312, viscose spinning and drawing 314, bleaching, cleaning, and drying 316 before final forming into artificial natural fiber filament yarns, stable fibers, etc. 318.

Thus, a system or product may include several different versions of disclosed compositions such as various encapsulated enzymes (e.g., stabilized enzymes), enzymes, engineered microbes, microbes, or others. Furthermore, a system described may produce many carbon products, byproducts (such as water), and other products and may be each processed further or combined further with other compounds to make artificial or novel materials or products.

In another embodiment, encapsulated enzymes or microbes are dried, packaged and/or embedded in a material, able to be used by individuals or entities to perform some of the related reactions or methods. These encapsulated enzymes may be packaged with a system or kit or some necessary inputs enabling the end user to operate it and perform the related processes. In some embodiments, operation includes an individual using the kit: adding water, the included encapsulated enzymes/microbes, included nutrients or adding external materials, and thus enabling the kit to operate and produce a desired product through reactions. The encapsulation as part of the disclosed composition helps the system operate in unregulated environments. One example of operation includes the continuous or batched production of edible proteins. In another embodiment, the encapsulated enzymes/microbes are immobilized on a surface or in a material to allow for easy product separation and active compound retention.

Industrial Use of Process with Carbon Dioxide Producing Facility

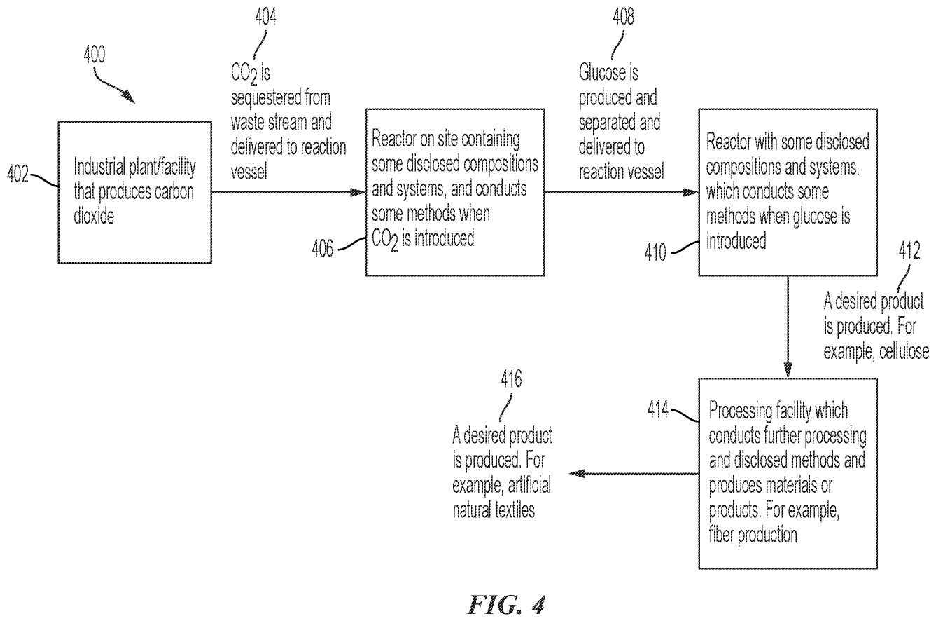

With reference to FIG. 4, exemplary process 400 depicts the production of glucose and biological macromolecules using stabilized enzymes from carbon dioxide produced by an industrial plant or facility 402. In some embodiments, an on-site reactor containing stabilized enzymes for various pathways (e.g., the Calvin Cycle and Gluconeogenesis) receives the carbon dioxide produced by the industrial plant or facility 402 to produce glucose 408. In some embodiments, carbon dioxide is sequestered from a waste stream of an industrial plant and delivered to a reaction vessel 404. In some embodiments, the reactor for producing biological macromolecules in on-site and uses stabilized enzymes as described herein to perform a variety of reactions as described herein 406. In some embodiments, biological macromolecules are produced from the glucose by stabilized enzymes 410. In some embodiments, the biological macromolecules are cellulose 412. In certain variations, the biological macromolecules are further processed 414 into desired useful products 416. In some variations, “CO₂” and “carbon dioxide” refer to any form of carbon dioxide.

### Stabilized Enzymes

In some embodiments, “stabilized enzyme” refers to enzymes that are (1) stabilized with surface-complementary intrinsically disordered polymer chains, (2) immobilized through adsorption and cross-linking, e.g., on non-self-assembling, micro or macro polymeric surfaces (e.g., microbeads or resin surface), and/or (3) stabilized through cross-linking the enzymes with themselves or other molecules. In exemplary embodiments, the stabilized enzyme is stable and active across varying temperature ranges, pH ranges, and/or robust industrial process time scales. In other exemplary embodiments, the stabilized enzyme is stable and active in an aqueous environment.

In some variations, the stabilized enzyme is in the form of an encapsulated enzyme, as disclosed in the compositions and embodiments herein. In certain variations, the stabilized enzymes are immobilized in a reaction vessel (e.g., through polymer structure immobilization) to enable easy separation of product.

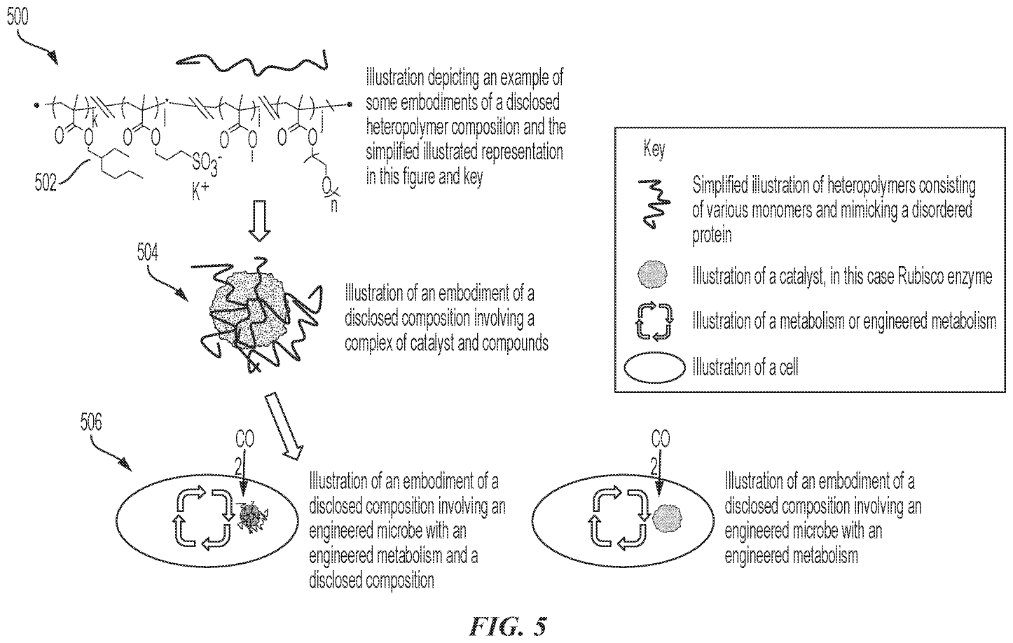

FIG. 5 depicts an exemplary process 500 of making and using exemplary stabilized enzymes. In step 502, depicted is an example of a heteropolymers composition, where the line is a simplified illustration of heteropolymers comprising, or consisting of, various monomers that mimic a disordered protein. In step 504, the heteropolymers composition is combined with an exemplary catalyst, in this case RuBiSCO enzyme, resulting in a composition that involves a complex of catalyst and compounds. In step 506, the RuBiSCO enzyme encapsulated by the heteropolymers composition is depicted in the use of an exemplary cell. Step 506 illustrates both an embodiment of a disclosed composition involving an engineered microbe with an engineered metabolism; as well as an embodiment of a disclosed composition involving an engineered microbe with an engineered metabolism and the RuBiSCO enzyme encapsulated by the heteropolymers composition.

In one aspect, provided is a composition comprising or consisting essentially of a complex of a catalyst and compounds where the composition mitigates negative impacts of its environment on activity or longevity of the catalyst if it were not complexed in said environment. In some embodiments, the complex of compounds and catalyst enables catalyst activity and longevity in various non-native environments.

In some embodiments, heteropolymers are polymerized through radical polymerization from monomers such as methacrylate-based monomers (for example methyl methacrylate (MMA), oligo(ethylene glycol) methacrylate (OEGMA), 3-sulfopropyl methacrylate potassium salt (3-SPMA), 2-ethylhexyl methacrylate (2-EHMA)) in a ratio resembling a naturally disordered protein, or in such a ratio to achieve desired properties of the disclosed composition. The monomers are selected to optimize short-range polymer-enzyme interactions (such as interacting with hydrophobic, positively charged, etc regions of the enzyme surface) and provide chemical diversity. The selection of monomer ratios are guided by solubility parameters to achieve the best retention in enzyme activity. The heteropolymers may have a number-average molecular weight on the order of 30-100 kDa, or about 40 kDa.

In some embodiments, the stabilized enzymes are produced by mixing the enzyme and compounds in an aqueous solution; drying the mixture; and resuspending the dried mixture in a solution, forming the composition. In some embodiments, the stabilized enzymes are produced by essentially of mixing the enzyme and compounds in a media which allows high enzyme activity such that they form a complex; and drying the mixture, forming the composition. In some embodiments, the stabilized enzymes are produced by mixing the enzyme and compounds in a solution which allows high enzyme activity such that they form the composition.

In some embodiments, the heteropolymers are mixed with the RuBisCO enzyme to encapsulate the enzyme. The mixture is dried then resuspended in a desired solvent with necessary reactants such as ribulose 1,5-bisphosphate (“RuBP”) as well as electron donors (such as NADPH and/or nicotinamide adenine dinucleotide (“NADH”), the reduced form of NADPH) and energy molecules (such as ATP). With the encapsulation and in this solvent, the encapsulated enzyme maintains or improves activity or longevity partially due to the encapsulation without having to strictly regulate the solution pH or temperature. In some embodiments, the encapsulated enzyme is able to resist conformational change and protect enzyme activity in a non-native environment through the polymers adjusting their conformations to maximize enzyme-polymer interactions in any solvent. In an example of evaluating retention of enzyme activity of a disclosed encapsulated enzyme, the composition is dispersed in solution to perform colorimetric assay. In another example of evaluating retention of enzyme activity of a disclosed encapsulated enzyme, the composition is dispersed in solution to perform an activity assay.

In some embodiments, the heteropolymers of the stabilized enzymes mimic naturally disordered proteins. In some embodiments, the enzymes of the stabilized enzymes are enzymes for producing glyceraldehyde 3-phosphate from carbon dioxide via the regenerative Calvin Cycle as described herein. In some embodiments, the enzymes of the stabilized enzymes are enzymes for producing glucose from glyceraldehyde 3-phosphate via the gluconeogenesis pathway as described herein. In some embodiments, the enzymes of the stabilized enzymes are enzymes for producing cellulose from glucose via various enzymes required for the synthesis of cellulose as described herein. In some embodiments, the enzymes of the stabilized enzymes are enzymes for producing starch from glucose via various enzymes required for the synthesis of starch as described herein.

In some embodiments, the heteropolymers are mixed with numerous enzymes (e.g., Calvin Cycle enzymes, gluconeogenesis enzymes, and enzymes required to produce cellulose from glucose) to encapsulate the various enzymes simultaneously. For example, in certain variations, the Calvin Cycle enzymes as described herein (e.g., RuBisCO, phosphoglycerate kinase, etc.) are mixed with heteropolymers to produce encapsulated (e.g., stabilized) Calvin Cycle enzymes. In some variations, Calvin Cycle enzymes and gluconeogenesis enzymes are mixed with heteropolymers to produce a mixture of stabilized Calvin Cycle enzymes and stabilized gluconeogenesis enzymes. In other variations, Calvin Cycle enzymes, gluconeogenesis enzymes, and enzymes for synthesizing cellulose from glucose are mixed with heteropolymers to produce a mixture of stabilized Calvin Cycle enzymes, stabilized gluconeogenesis enzymes, and stabilized enzymes for synthesizing cellulose from glucose. In other variations, Calvin Cycle enzymes, gluconeogenesis enzymes, and enzymes for synthesizing starch from glucose are mixed with heteropolymers to produce a mixture of stabilized Calvin Cycle enzymes, stabilized gluconeogenesis enzymes, and stabilized enzymes for synthesizing starch from glucose.

### Carbon Source

The carbon dioxide used in the systems and methods herein may be obtained from any commercially available source or obtained using any methods known in the art. In some variations, the production system includes a tank containing carbon dioxide that feeds into the reactor. In some variations, the reactor in the production systems herein is positioned on-site of a carbon dioxide-producing facility (e.g., a direct air capturing facility) or a carbon dioxide-capturing facility, and the CO₂ is delivered to said reactor (e.g., FIG. 4). A disclosed composition, reactants, and electron donors are present in said reactor in solution. CO₂ is converted into a carbon product through a continuous process on-site. Due to the disclosed composition's encapsulation, the reaction vessel and reaction do not require significant regulation in terms of temperature, pH, or pressure, whereas an uncomplexed enzyme would.

In other variations, CO₂ from an industrial facility's waste stream is captured and stored in metal organic frameworks (MOFs). The MOFs are introduced into a reaction vessel and heated to release the CO₂. The released CO₂ is used as an input to a disclosed carbon fixation system and method as described herein. In another variation, a metal organic framework device is fixed within a vehicle exhaust or ambient source of CO₂ in order to collect CO₂ molecules, and is then heated to release CO₂ in a chamber with a disclosed composition to fix the carbon dioxide instead of being emitted into the air.

In other variations, the composition, systems, and methods are applied to a carbon source such as forms of inorganic carbon and/or C1 carbon sources including carbon monoxide, methane, methanol, formate, or formic acid, and/or mixtures containing C1 chemicals including various syngas compositions, into organic chemicals. In another variation, any combination of disclosed systems, compositions, and/or methods exist on Mars and use CO₂ from the Martian atmosphere as an input.

### Production Systems

In some aspects, provided is a production system for producing biological macromolecules from carbon dioxide using a an enzymatic cascade of stabilized enzymes for converting carbon dioxide a desired product (e.g., biological macromolecules and various intermediates such as glucose, cellulose, and starch). In some embodiments, the production system includes a reusable system of enzymes (e.g., the Calvin Cycle), regenerating inputs (e.g., ribulose 1,5-bisphosphate), and regenerating energy/electron sources (e.g., adenosine triphosphate, nicotinamide adenine dinucleotide phosphate, and uridine triphosphate). In some embodiments, provided is a production system for producing glyceraldehyde 3-phosphate from carbon dioxide using stabilized Calvin Cycle enzymes as described herein. In some embodiments, provided is a production system for producing glucose from glyceraldehyde 3-phosphate from using stabilized gluconeogenesis enzymes as described herein. In some embodiments, provided is a production system for producing cellulose from glucose from using various stabilized enzymes required for the synthesis of cellulose from glucose as described herein. In some embodiments, provided is a production system for producing starch from glucose from using various stabilized enzymes required for the synthesis of starch from glucose as described herein. In some variations, inputs into the systems and methods, such as substrates, enzymes, may be provided from lysed cells (e.g. plant, bacterial, yeast cells).

### “G3P” Production

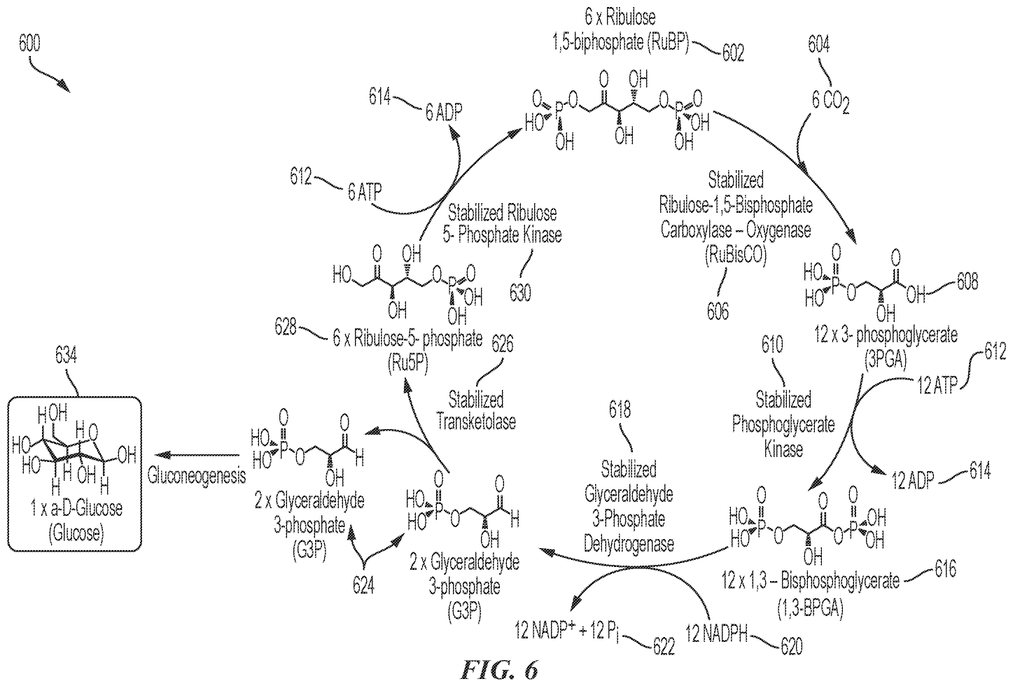

FIG. 6 depicts the regenerative Calvin Cycle 600 with stabilized enzymes described herein for producing glyceraldehyde-3-phosphate (“G3P”) 622 from carbon dioxide 604. In some embodiments, the production system produces G3P 622 from carbon dioxide 604 via the regenerative Calvin Cycle 602 using stabilized Calvin Cycle enzymes. In FIG. 6, stabilized RuBisCO 606 catalyzes a reaction between ribulose RuBP 602 and carbon dioxide 604 to produce 3-phosphoglyceric acid (3-PGA) 608; stabilized phosphoglycerate kinase 610 converts 3-PGA to 1,3-bisphosphoglyceric acid (1,3-BPGA) 616 using energy from adenosine triphosphate (ATP) 612, which is converted into adenosine diphosphate (ADP) 614; stabilized glyceraldehyde 3-phosphate dehydrogenase 618 converts 1,3-BPGA to G3P 622 using an electron donating source; stabilized transketolase 626, ribulose-5-phosphate kinase 630, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, and stabilized phosphoribulokinase convert G3P 622 to ribulose-5-phosphate (Ru5P) 628 and Ru5P 628 into RuBP 620 using ATP 612, thereby regenerating RuBP 602. The regeneration of RuBP 602 regenerates the Calvin Cycle. In FIG. 6, the electron donating source is nicotinamide adenine dinucleotide phosphate (“NADPH”) 620, which is converted into NADP+ 622, the oxidized form of NADPH. In some embodiments, G3P 622 is further converted into glucose 634 via the gluconeogenesis pathway. In some embodiments, the electron donating source is an electrode.

Stabilized ATP Regenerating Enzyme

In some embodiments, the production system for producing G3P comprises a stabilized ATP regenerating enzyme. The stabilized ATP regenerating enzyme regenerates ATP in the reactor using the phosphate source. In some embodiments, the ATP regenerating enzyme is a stabilized kinase enzyme. In certain embodiments, the ATP regenerating enzyme is a stabilized polyphosphate kinase enzyme. In some embodiments, the phosphate source is polyphosphate. In some embodiments, the production system is configured to recycle the phosphate agent. In such embodiments, the phosphate agent is present in the reaction prior to starting the reaction to produce biological macromolecules or intermediates as described herein.

Electron Donating Source

In some embodiments, the electron donating source is NADPH. In some embodiments, the electron donating source is NADH. In some embodiments, the reactor receives NAPDH and/or NADH from a NADPH and/or NADH source configured to output NADPH and/or NADH into the reactor. In other embodiments, the reactor contains NADPH and/or NADH before the reaction begins. In some embodiments, the production system comprises a stabilized NAPDH regenerating enzyme. The stabilized NADPH and/or NADH regenerating enzyme regenerates NADPH and/or NADH in the reactor using the electron donating source. In some variations, the NADPH and/or NADH regenerating enzyme is a stabilized hydrogenase enzyme. In certain variations, the NADPH and/or NADH regenerating enzyme is a stabilized glucose dehydrogenase enzyme.

In other embodiments, the electron donating source described herein is an electron source. In such embodiments, the electrons are delivered to the reaction through an electrode, electricity source, electrochemical source, or ion source. In certain embodiments, the electron source is located in the reactor and is configured to provide electrons to the aqueous media.

CO2 to G3P—Regeneration of the Calvin Cycle and Regeneration of ATP

In some aspects, provided is a production system to produce G3P from carbon dioxide via the Calvin Cycle. In some embodiments, the production system for producing G3P includes: a carbon dioxide source configured to output carbon dioxide; a phosphate source configured to output a phosphate agent; and a reactor configured to receive carbon dioxide from the carbon dioxide source and the phosphate agent from the phosphate source into the reactor containing RuBP, stabilized RuBisCO, ATP, a stabilized ATP regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media. The reactor is configured to: (i) produce G3P, (ii) regenerate RuBP, and (iii) regenerate ATP.

G3P Production—RuBP Source and Regeneration of ATP

In some aspects, provided is a production system for producing G3P from carbon dioxide using a RuBP source configured to output RuBP into the reactor. In some embodiments, the production system for producing G3P from carbon dioxide using a RuBP source includes: a carbon dioxide source configured to output carbon dioxide; a phosphate source configured to output a phosphate agent; a RuBP source configured to output RuBP; and a reactor configured to receive carbon dioxide from the carbon dioxide source, phosphate agent from the phosphate source, and RuBP from the RuBP source into the reactor containing stabilized RuBisCO, ATP, a stabilized ATP regenerating enzyme, stabilized phosphoglycerate kinase, stabilized glyceraldehyde 3-phosphate dehydrogenase, and an electron donating source in an aqueous media. The reactor is configured to: (i) produce G3P and (ii) regenerate ATP.

G3P Production—ATP Source and Regeneration of the Calvin Cycle

In some aspects, provided is a production system for producing G3P from carbon dioxide using an ATP source configured to output ATP into the reactor. In some embodiments, the production system for producing G3P from carbon dioxide using an ATP source includes: a carbon dioxide source configured to output carbon dioxide; an ATP source configured to output ATP; and a reactor configured to receive carbon dioxide from the carbon dioxide source and ATP from the ATP source into the reactor containing RuBP, stabilized RuBisCo, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in aqueous media. The reactor is configured to: (i) produce G3P and (ii) regenerate RuBP.

### Glucose Production

FIG. 7 depicts the gluconeogenesis pathway 700 with stabilized enzymes described herein for producing glucose 634 from G3P 624. Glucose 634 is produced from G3P 624 by a series of reactions catalyzed by stabilized aldolase 702, stabilized fructose 1,6-bisphosphatase 706, stabilized phosphoglucose isomerase 712, and stabilized glucose 6-phosphatase 716. In FIG. 7, stabilized aldolase 702 converts G3P 624 to fructose 1,6-bisphosphate 704; stabilized fructose 1,6-bisphosphatase 706 converts fructose 1,6-bisphosphate 704 to fructose 6-phosphate 710 using water 708; stabilized phosphoglucose isomerase 712 converts fructose 6-phosphate 710 to glucose 6-phosphate 714; stabilized glucose 6-phosphatase 716 converts glucose 6-phosphate 714 to glucose 634 using water 708. In some embodiments, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, and stabilized pyruvate carboxylase are also present for synthesizing glucose from G3P via the gluconeogenesis pathway.

CO2 to Glucose—Regeneration of the Calvin Cycle and Regeneration of ATP

In some aspects, provided is a production system for producing glucose from carbon dioxide via the Calvin Cycle and gluconeogenesis pathway. In some embodiments, the production system for producing glucose from carbon dioxide includes: a carbon dioxide source configured to output carbon dioxide; a phosphate source configured to output a phosphate agent; a water source configured to output water; and a reactor configured to receive carbon dioxide from the carbon dioxide source, the phosphate agent from the phosphate source, and water from the water source, into the reactor containing RuBP, stabilized RuBisCo, ATP, a stabilized ATP regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, and stabilized pyruvate carboxylase, and an electron donating source in an aqueous media. The reactor is configured to (i) produce G3P, (ii) regenerate RuBP, (iii) regenerate ATP, and (iv) convert G3P to glucose.

Stabilized ATP Regenerating Enzyme

In some embodiments, the production system for producing glucose comprises a stabilized ATP regenerating enzyme. The stabilized ATP regenerating enzyme regenerates ATP in the reactor using the phosphate agent. In some embodiments, the ATP regenerating enzyme is a stabilized kinase enzyme. In certain embodiments, the ATP regenerating enzyme is a stabilized polyphosphate kinase. In some embodiments, the phosphate agent is polyphosphate. In some embodiments, the production system is configured to recycle the phosphate agent. In such embodiments, the phosphate agent is present in the reaction prior to starting the reaction to produce biological macromolecules or intermediates as described herein.

Electron Donating Source

In some embodiments, the electron donating source is NADPH. In some embodiments, the electron donating source is NADH. In some embodiments, the reactor receives NAPDH and/or NADH from a NADPH and/or NADH source configured to output NADPH and/or NADH into the reactor. In other embodiments, the reactor contains NADPH and/or NADH before the reaction begins. In some embodiments, the production system comprises a stabilized NAPDH regenerating enzyme. The stabilized NADPH and/or NADH regenerating enzyme regenerates NADPH and/or NADH in the reactor using the electron donating source. In some variations, the NADPH and/or NADH regenerating enzyme is a stabilized hydrogenase enzyme. In certain variations, the NADPH and/or NADH regenerating enzyme is a stabilized glucose dehydrogenase enzyme.

In other embodiments, the electron donating source described herein is an electron source. In such embodiments, the electrons are delivered to the reaction through an electrode, electricity source, electrochemical source, or ion source. In certain embodiments, the electron source is located in the reactor and is configured to provide electrons to the aqueous media.

CO2 to Glucose—RuBP Source and Regeneration of ATP

In some embodiments, the production system for producing glucose from carbon dioxide comprises a RuBP source configured to output RuBP into the reactor (i.e., the production system does not regenerate RuBP using a stabilized RuBP regenerating enzyme). For example, in some variations, the production system for producing glucose from carbon dioxide comprises a RuBP source configured to output RuBP for producing G3P from carbon dioxide as described herein.

CO2 to Glucose—ATP Source and Regeneration of the Calvin Cycle

In some embodiments, the production system for producing glucose from carbon dioxide comprises an ATP source configured to output ATP into the reactor (i.e., the production system does not regenerate ATP using a stabilized ATP regenerating enzyme). For example, in such variations, the production system for producing glucose from carbon dioxide comprises an ATP source configured to output ATP for producing G3P from carbon dioxide as described herein.

In some variations, the production system comprises a single reactor for producing G3P from carbon dioxide and converting G3P to glucose. In other variations, the production system comprises a first reactor and a second reactor. In such variations, the first reactor is configured to produce G3P from carbon dioxide and to output G3P to the second reactor and the second reactor is configured to receive the G3P from the first reactor and convert G3P to glucose.

G3P to Glucose

In some aspects, provided is a production system to produce glucose from G3P via the gluconeogenesis pathway. In some embodiments, the production system for producing glucose from G3P includes: a G3P source configured to output G3P; a water source configured to output water; and a reactor configured to receive G3P from the G3P source, and water from the water source into the reactor containing stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, and stabilized pyruvate carboxylase in an aqueous media. The reactor is configured to produce glucose.

In some variations of the foregoing, the systems and methods described above may be performed in one or multiple reactors. For example, in some embodiments, the production system comprises a first reactor configured to produce G3P and output G3P to a second reactor, and the second reactor is configured to convert G3P to glucose using various stabilized enzymes for gluconeogenesis as described herein. In other embodiments, the production system comprises a first reactor configured to produce a first product .e.g, any of the intermediates for converting carbon dioxide to glucose such as G3P, fructose 1,6-bisphosphate, fructose 6-phosphate, etc.) using stabilized enzymes as described herein and output the first product to a second reactor; the second reactor is configured to convert the first product into a second product (e.g., glucose or intermediates) using stabilized enzymes as described herein; and an optional third reactor configured to produce a third product using various stabilized enzymes as described herein when the second product is an intermediate product for the synthesis of glucose (e.g., G3P, fructose 1,6-bisphosphate, etc.).

### Cellulose Production

In an exemplary embodiment, the production system produces cellulose from carbon dioxide via the Calvin Cycle and the gluconeogenesis pathway. In such an exemplary embodiment the production system: converts carbon dioxide to G3P using various inputs, such as electrons, RuBP, and stabilized Calvin Cycle enzymes as described herein; converts G3P to glucose using various inputs, such as G3P produced via the Calvin Cycle, water, and various stabilized gluconeogenesis enzymes as described herein; and produces cellulose using various inputs, such as glucose produced via the gluconeogenesis pathway ATP, uridine triphosphate (“UTP”), and various enzymes for synthesizing cellulose as described herein.

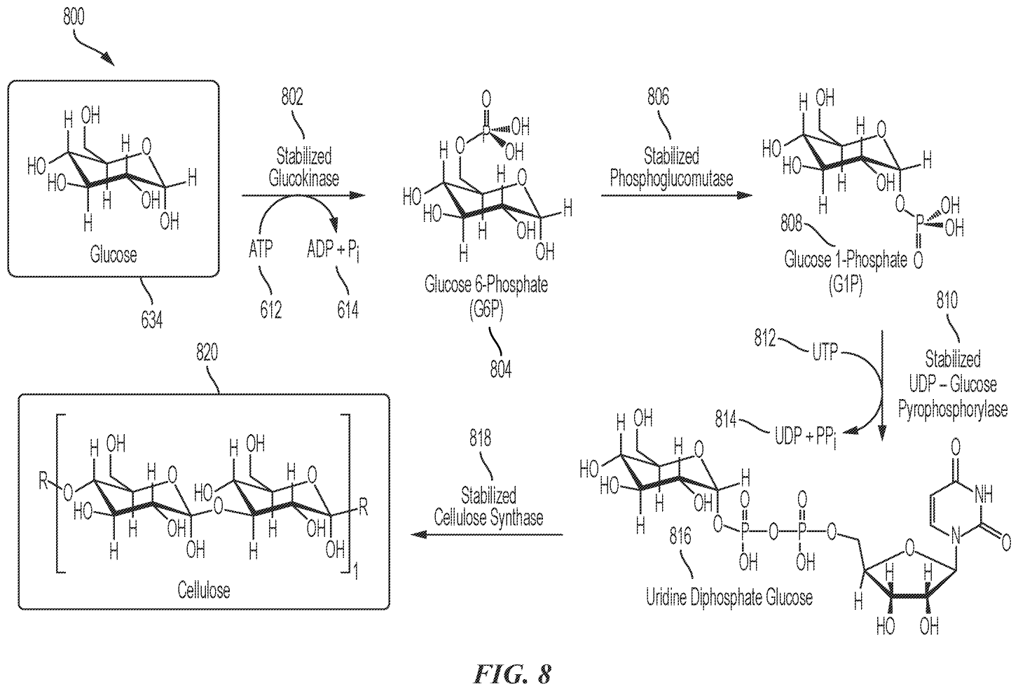

FIG. 8 depicts the cellulose synthesis pathway 800 which produces cellulose 820 from glucose 634 using various stabilized enzymes described herein. Cellulose 820 is produced by series of reactions catalyzed by stabilized glucokinase 802, stabilized phosphoglucomutase 806, stabilized glucose-1-phosphate uridylyltransferase 810, and stabilized starch synthase 818. In FIG. 8, stabilized glucokinase 802, converts glucose 634 to glucose 6-phosphate 804 using ATP 612, which is converted into ADP 612; stabilized phosphoglucomutase 806 converts glucose 6-phosphate 804 to glucose 1-phosphate 808; stabilized glucose-1-phosphate uridylyltransferase 810 converts glucose 1-phosphate 808 to uridine diphosphate glucose 816 UTP 812, where UTP 812 is converted to uridine diphosphate (“UDP”) 814; and stabilized cellulose synthase 818 converts uridine diphosphate glucose 816 to cellulose 820.

Stabilized ATP Regenerating Enzyme

In some embodiments, the production system for producing cellulose comprises a stabilized ATP regenerating enzyme. The stabilized ATP regenerating enzyme regenerates ATP in the reactor using the phosphate agent. In some embodiments, the ATP regenerating enzyme is a stabilized kinase enzyme. In certain embodiments, the ATP regenerating enzyme is a stabilized polyphosphate kinase. In some embodiments, the phosphate agent is polyphosphate. In some embodiments, the production system is configured to recycle the phosphate agent. In such embodiments, the phosphate agent is present in the reaction prior to starting the reaction to produce biological macromolecules or intermediates as described herein.

Stabilized UTP Regenerating Enzyme

In some embodiments, the production system for producing cellulose comprises a stabilized UTP regenerating enzyme. The stabilized UTP regenerating enzyme regenerates UTP in the reactor using the phosphate agent. In some embodiments, the UTP regenerating enzyme is a stabilized kinase enzyme. In certain embodiments, the UTP regenerating enzyme is a stabilized polyphosphate kinase enzyme. In some embodiments, the phosphate agent is polyphosphate. In some embodiments, the production system is configured to recycle the phosphate agent. In such embodiments, the phosphate agent is present in the reaction prior to starting the reaction to produce biological macromolecules or intermediates as described herein.

Electron Donating Source

In some embodiments, the electron donating source is NADPH. In some embodiments, the electron donating source is NADH. In some embodiments, the reactor receives NAPDH and/or NADH from a NADPH and/or NADH source configured to output NADPH and/or NADH into the reactor. In other embodiments, the reactor contains NADPH and/or NADH before the reaction begins. In some embodiments, the production system comprises a stabilized NAPDH regenerating enzyme. The stabilized NADPH and/or NADH regenerating enzyme regenerates NADPH and/or NADH in the reactor using the electron donating source. In some variations, the NADPH and/or NADH regenerating enzyme is a stabilized hydrogenase enzyme. In certain variations, the NADPH and/or NADH regenerating enzyme is a stabilized glucose dehydrogenase enzyme.

In other embodiments, the electron donating source described herein is an electron source. In such embodiments, the electrons are delivered to the reaction through an electrode, electricity source, electrochemical source, or ion source. In certain embodiments, the electron source is located in the reactor and is configured to provide electrons to the aqueous media.

CO2 to Cellulose—Regeneration of the Calvin Cycle and Regeneration of ATP

In some aspects, provided is a production system for producing cellulose from carbon dioxide via the Calvin Cycle and gluconeogenesis pathway. In some embodiments, the production system for producing cellulose from carbon dioxide includes: a carbon dioxide source configured to output carbon dioxide; a phosphate source configured to output a phosphate agent; a water source configured to output water; and a reactor configured to: receive carbon dioxide from the carbon dioxide source, the phosphate agent from the phosphate source, and water from the water source into the main reactor containing RuBP, stabilized RuBisCo, ATP, a stabilized ATP regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, UTP, a stabilized UTP regenerating enzyme, stabilized glucokinase, stabilized phosphoglucomutase, stabilized glucose-1-phosphate uridylyltransferase, stabilized cellulose synthase, and an electron donating agent in an aqueous media. The reaction is configured to: (i) produce G3P, (ii) regenerate RuBP, (iii) regenerate ATP, (iv) convert G3P to glucose, (v) convert glucose to cellulose, and (vi) regenerate UTP.

CO2 to Cellulose—RuBP Source and Regeneration of ATP

In some embodiments, the production system for producing cellulose from carbon dioxide comprises a RuBP source configured to output RuBP into the reactor (i.e., the production system does not regenerate RuBP using stabilized RuBP regenerating enzymes). For example, in some variations, the production system for producing cellulose from carbon dioxide comprises a RuBP source configured to output RuBP for producing G3P from carbon dioxide as described herein.

CO2 to Cellulose—ATP Source and Regeneration of the Calvin Cycle

In some embodiments, the production system for producing cellulose from carbon dioxide comprises an ATP source configured to output ATP into the reactor (i.e., the production system does not regenerate ATP using a stabilized ATP regenerating enzyme). For example, in such variations, the production system for producing cellulose from carbon dioxide comprises an ATP source configured to output ATP for producing G3P from carbon dioxide as described herein.

In some embodiments, the production system for producing cellulose from carbon dioxide comprises a single reactor for producing G3P from carbon dioxide, glucose from G3P, and cellulose from glucose. In some variations, the production system produces G3P from carbon dioxide, glucose from G3P, and cellulose from glucose concurrently (e.g., at the same time). In other variations, the production system produces G3P from carbon dioxide, glucose from G3P, and cellulose from glucose sequentially (e.g., step-wise).

In some embodiments, the production system for producing cellulose from carbon dioxide comprises a first reactor, a second reactor, and a third reactor. In such variations, the first reactor is configured to produce G3P from carbon dioxide and to output G3P to the second reactor; the second reactor is configured to receive the G3P from the first reactor and convert G3P to glucose; and the third reactor is configured to receive the glucose from the second reactor and covert glucose to cellulose. In other variations, the production system comprises a first reactor and a second reactor configured such that the first reactor is configured to produce G3P from carbon dioxide and glucose from G3P and the second reactor is configured to produce cellulose from glucose. In another variation, the production system comprises a first reactor and a second reactor configured such that the first reactor is configured to produce of G3P from carbon dioxide and the second reactor is configured to produce glucose from G3P and cellulose from glucose.

G3P to Cellulose

In some aspects, provided is a production system for producing cellulose from G3P via the gluconeogenesis pathway. In some embodiments, the production system for producing cellulose from G3P includes: a G3P source configured to output G3P; a phosphate source configured to output a phosphate agent; a water source configured to output water; and a reactor configured to receive G3P from the G3P source, the phosphate agent from the phosphate source, and water from the water source into the reactor containing stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, ATP, a stabilized ATP regenerating enzyme, UTP, a stabilized UTP regenerating enzyme, stabilized glucokinase, stabilized phosphoglucomutase, stabilized glucose-1-phosphate uridylyltransferase, and stabilized cellulose synthase in an aqueous media. The reaction is configured to: (i) convert G3P to glucose, (ii) convert glucose to cellulose, (iii) regenerate ATP, and (iv) regenerate UTP.

Glucose to Cellulose

In some aspects, provided is a production system for producing cellulose from glucose. In some embodiments, the production system for producing cellulose from glucose includes: a glucose source configured to output glucose; a phosphate source configured to output a phosphate agent; and a reactor configured to receive glucose from the glucose source and the phosphate agent from the phosphate source into the reactor containing ATP, a stabilized ATP regenerating enzyme, UTP, a stabilized UTP regenerating enzyme, stabilized glucokinase, stabilized phosphoglucomutase, stabilized glucose-1-phosphate uridylyltransferase, stabilized cellulose synthase, and an electron donating agent in aqueous media. The reaction is configured to: (i) convert glucose to cellulose, (ii) regenerate ATP, and (iii) regenerate UTP.

In some variations of the foregoing, the systems and methods described above may be performed in one or multiple reactors. For example, in some embodiments, the production system comprises a first reactor configured to produce G3P and output G3P to a second reactor; the second reactor is configured to convert G3P to glucose using various stabilized enzymes for gluconeogenesis as described herein and to output glucose to a third reactor; the third reactor is configured to convert glucose to cellulose using various stabilized enzymes for the synthesis of cellulose as described herein. In other embodiments, the production system comprises a first reactor configured to produce a first product (e.g, any of the intermediates for converting carbon dioxide to cellulose such as G3P, fructose 1,6-bisphosphate, fructose 6-phosphate, glucose, glucose 1-phosphate, etc.) and output the first product to a second reactor; the second reactor is configured to convert the first product into a second product (e.g., cellulose or intermediates); and an optional third reactor configured to produce a third product when the second product is an intermediate product for the synthesis of cellulose (e.g., G3P. fructose 1,6-bisphosphate, glucose 1-phosphate, etc.).

Methods for Producing Biological Macromolecules and Intermediates from Carbon Dioxide

In some aspects, provided are methods for producing biological macromolecules and intermediates thereof from carbon dioxide using stabilized enzymes using the production systems described herein.

Methods for Producing G3P

In one aspect, provided is a method for producing glyceraldehyde 3-phosphate from carbon dioxide using stabilized enzymes, comprising: combining carbon dioxide, a phosphate agent, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media; producing glyceraldehyde 3-phosphate; and regenerating ribulose 1,5-bisphosphate and adenosine triphosphate. In some variations, the stabilized adenosine triphosphate regenerating enzyme comprises a kinase. In other variations, the stabilized adenosine triphosphate regenerating enzyme comprises a polyphosphate kinase. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments, the method for producing glyceraldehyde 3-phosphate from carbon dioxide using stabilized enzymes further comprises recycling the phosphate agent.

In another aspect, provided is a method for producing glyceraldehyde 3-phosphate from carbon dioxide using stabilized enzymes, comprising: combining carbon dioxide, a phosphate agent, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, and an electron donating source in an aqueous media; producing glyceraldehyde 3-phosphate; and regenerating adenosine triphosphate. In some variations, the stabilized adenosine triphosphate regenerating enzyme comprises a kinase. In other variations, the stabilized adenosine triphosphate regenerating enzyme comprises a polyphosphate kinase. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments, the method for producing glyceraldehyde 3-phosphate from carbon dioxide using stabilized enzymes further comprises recycling the phosphate agent.

In another aspect, provided is a method for producing glyceraldehyde 3-phosphate from carbon dioxide using stabilized enzymes, comprising: combining carbon dioxide, adenosine triphosphate, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media; producing glyceraldehyde 3-phosphate; and regenerating ribulose 1,5-bisphosphate. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode.

Methods for Producing Glucose

In one aspect, provided is a method for producing glucose from carbon dioxide using stabilized enzymes, comprising: combining carbon dioxide, a phosphate agent, water, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating agent in an aqueous media; producing glyceraldehyde 3-phosphate; regenerating ribulose 1,5-bisphosphate and adenosine triphosphate; and converting glyceraldehyde 3-phosphate to glucose. In some variations, the stabilized adenosine triphosphate regenerating enzyme comprises a kinase. In other variations, the stabilized adenosine triphosphate regenerating enzyme comprises a polyphosphate kinase. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments, the method for producing glucose from carbon dioxide using stabilized enzymes further comprises recycling the phosphate agent.

In another aspect, provided is a method for producing glucose from carbon dioxide using stabilized enzymes, comprising: combining carbon dioxide, ribulose 1,5-bisphosphate, a phosphate agent, water, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media; producing glyceraldehyde 3-phosphate; regenerating adenosine triphosphate; and converting glyceraldehyde 3-phosphate to glucose. In some variations, the stabilized adenosine triphosphate regenerating enzyme comprises a kinase. In other variations, the stabilized adenosine triphosphate regenerating enzyme comprises a polyphosphate kinase. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments, the method for producing glucose from carbon dioxide using stabilized enzymes further comprises recycling the phosphate agent.

In another aspect, provided is a method for producing glucose from carbon dioxide using stabilized enzymes, comprising: combining carbon dioxide, adenosine triphosphate, water, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media; producing glyceraldehyde 3-phosphate; regenerating ribulose 1,5-bisphosphate; and converting glyceraldehyde 3-phosphate to glucose. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode.

In another aspect, provided is a method for producing glucose from glyceraldehyde 3-phosphate using stabilizing enzymes, comprising: combining glyceraldehyde 3-phosphate, water, stabilized aldolase, stabilized fructose 1,6-bisphosphatase, stabilized phosphoglucose isomerase, and stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase in an aqueous media; and producing glucose.

Methods for Producing Cellulose

In another aspect, provided is a method for producing cellulose from carbon dioxide using stabilized enzymes, comprising: combining carbon dioxide, a phosphate agent, water, ribulose-1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media; producing glyceraldehyde 3-phosphate; regenerating ribulose 1,5-bisphosphate and adenosine triphosphate, and uridine triphosphate; and converting glyceraldehyde 3-phosphate to glucose and glucose to cellulose. In some variations, the stabilized adenosine triphosphate regenerating enzyme comprises a kinase. In other variations, the stabilized adenosine triphosphate regenerating enzyme comprises a polyphosphate kinase. In some variations, the stabilized uridine triphosphate regenerating enzyme comprises a kinase. In other variations, the stabilized uridine triphosphate regenerating enzyme comprises a polyphosphate kinase. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments, the method for producing cellulose from carbon dioxide using stabilized enzymes further comprises recycling the phosphate agent.

In other variations, provided is a method for producing cellulose from carbon dioxide using stabilized enzymes, comprising: combining carbon dioxide, ribulose 1,5-bisphosphate, a phosphate agent, water, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media; producing glyceraldehyde 3-phosphate; regenerating adenosine triphosphate and uridine triphosphate; and converting glyceraldehyde 3-phosphate to glucose and glucose to cellulose. In some variations, the stabilized adenosine triphosphate regenerating enzyme comprises a kinase. In other variations, the stabilized adenosine triphosphate regenerating enzyme comprises a polyphosphate kinase. In some variations, the stabilized uridine triphosphate regenerating enzyme comprises a kinase. In other variations, the stabilized uridine triphosphate regenerating enzyme comprises a polyphosphate kinase. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments, the method for producing cellulose from carbon dioxide using stabilized enzymes further comprises recycling the phosphate agent.

In another aspect, provided is a method for producing cellulose from carbon dioxide using stabilized enzymes, comprising: combining carbon dioxide, a phosphate reagent, water, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media; producing glyceraldehyde 3-phosphate; regenerating ribulose 1,5-bisphosphate and uridine triphosphate; and converting glyceraldehyde 3-phosphate to glucose and glucose to cellulose. In some variations, the stabilized uridine triphosphate regenerating enzyme comprises a kinase. In other variations, the stabilized uridine triphosphate regenerating enzyme comprises a polyphosphate kinase. In some embodiments, the electron donating source comprises nicotinamide adenine dinucleotide phosphate or a reduced form of nicotinamide adenine dinucleotide phosphate, or any combination thereof. In such embodiments, a stabilized glucose dehydrogenase enzyme for regenerating nicotinamide adenine dinucleotide phosphate or the reduced form of nicotinamide adenine dinucleotide phosphate, or any combination thereof, may also be combined. In other embodiments, the electron donating source is an electrode. In some embodiments, the phosphate agent comprises polyphosphate. In certain embodiments, the method for producing cellulose from carbon dioxide using stabilized enzymes further comprises recycling the phosphate agent.

In another aspect, provided is a method for producing cellulose from glyceraldehyde 3-phosphate using stabilizing enzymes, comprising: combining glyceraldehyde 3-phosphate, a phosphate agent, water, adenosine triphosphate, a stabilized adenosine triphosphate regenerating enzyme, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, stabilized phosphoglucose isomerase, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media; converting glyceraldehyde 3-phosphate to glucose; regenerating adenosine triphosphate and uridine triphosphate; and converting glucose to cellulose. In some variations, the stabilized adenosine triphosphate regenerating enzyme comprises a kinase. In other variations, the stabilized adenosine triphosphate regenerating enzyme comprises a polyphosphate kinase. In some variations, the stabilized uridine triphosphate regenerating enzyme comprises a kinase. In other variations, the stabilized uridine triphosphate regenerating enzyme comprises a polyphosphate kinase. In some embodiments, the phosphate agent comprises polyphosphate. In certain embodiments, the method for producing cellulose from glyceraldehyde 3-phosphate using stabilized enzymes further comprises recycling the phosphate agent.

In another aspect, provided is a method for producing cellulose from glucose using stabilizing enzymes, comprising: combining glucose, a phosphate agent, adenosine triphosphate, a stabilized adenosine triphosphate regenerating enzyme, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media; converting glucose to cellulose; and regenerating adenosine triphosphate and uridine triphosphate. In some variations, the stabilized adenosine triphosphate regenerating enzyme comprises a kinase. In other variations, the stabilized adenosine triphosphate regenerating enzyme comprises a polyphosphate kinase. In some variations, the stabilized uridine triphosphate regenerating enzyme comprises a kinase. In other variations, the stabilized uridine triphosphate regenerating enzyme comprises a polyphosphate kinase. In some embodiments, the phosphate agent comprises polyphosphate. In certain embodiments, the method for producing cellulose from glucose using stabilized enzymes further comprises recycling the phosphate agent.

What is claimed is:

1. A production system for producing glyceraldehyde 3-phosphate from carbon dioxide, comprising:
a carbon dioxide source configured to output carbon dioxide;
a phosphate source configured to output a phosphate agent; and
a reactor configured to:
receive carbon dioxide from the carbon dioxide source and the phosphate agent from the phosphate source into the reactor containing ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media to:
(i) produce glyceraldehyde 3-phosphate,
(ii) regenerate ribulose 1,5-bisphosphate, and
(iii) regenerate adenosine triphosphate.

2. The production system of claim 1, wherein the stabilized adenosine triphosphate regenerating enzyme comprises a kinase.

3. The production system of claim 1, wherein the stabilized adenosine triphosphate regenerating enzyme comprises a polyphosphate kinase.

4. The production system of any one of claims 1 to 3, wherein the electron donating source comprises nicotinamide adenine dinucleotide phosphate or a reduced form of nicotinamide adenine dinucleotide phosphate, or any combination thereof.

5. The production system of claim 4, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

6. The production system of any one of claims 1 to 3, wherein the electron donating source is an electrode.

7. The production system of any one of claims 1 to 6, wherein the phosphate agent comprises polyphosphate.

8. A production system for producing glyceraldehyde 3-phosphate from carbon dioxide, comprising:
a carbon dioxide source configured to output carbon dioxide;
a phosphate source configured to output a phosphate agent;
a ribulose 1,5-bisphosphate configured to output ribulose 1,5-bisphosphate; and
a reactor configured to:
receive carbon dioxide from the carbon dioxide source, the phosphate agent from the phosphate source, and ribulose 1,5-bisphosphate from the ribulose 1,5-bisphosphate source into the reactor containing stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, and an electron donating source in an aqueous media to:
(i) produce glyceraldehyde 3-phosphate, and
(ii) regenerate adenosine triphosphate.

9. The production system of claim 8, wherein the stabilized adenosine triphosphate regenerating enzyme comprises a kinase.

10. The production system of claim 8, wherein the stabilized adenosine triphosphate regenerating enzyme comprises a polyphosphate kinase.

11. The production system of any one of claims 8 to 10, wherein the electron donating source comprises nicotinamide adenine dinucleotide phosphate or a reduced form of nicotinamide adenine dinucleotide phosphate, or any combination thereof.

12. The production system of claim 11, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

13. The production system of any one of claims 8 to 10, wherein the electron donating source is an electrode.

14. The production system of any one of claims 8 to 13, wherein the phosphate agent comprises polyphosphate.

15. A production system for producing glyceraldehyde 3-phosphate from carbon dioxide, comprising:
a carbon dioxide source configured to output carbon dioxide;
an adenosine triphosphate source configured to output adenosine triphosphate; and
a reactor configured to:
receive carbon dioxide from the carbon dioxide source and adenosine triphosphate from the adenosine triphosphate source into the reactor containing ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media to:
(i) produce glyceraldehyde 3-phosphate, and
(ii) regenerate ribulose 1,5-bisphosphate.

16. The production system of claim 15, wherein the electron donating source comprises nicotinamide adenine dinucleotide phosphate or a reduced form of nicotinamide adenine dinucleotide phosphate, or any combination thereof.

17. The production system of claim 16, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

18. The production system of any claim 15, wherein the electron donating source is an electrode.

19. A production system for producing glucose from carbon dioxide, comprising:
a carbon dioxide source configured to output carbon dioxide;
a phosphate source configured to output a phosphate agent;
a water source configured to output water; and
a reactor configured to:
receive carbon dioxide from the carbon dioxide source, the phosphate agent from the phosphate source, and water from the water source into the reactor containing ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating agent in an aqueous media to:
(i) produce glyceraldehyde 3-phosphate,
(ii) regenerate ribulose 1,5-bisphosphate,
(iii) regenerate adenosine triphosphate, and
(iv) convert glyceraldehyde 3-phosphate to glucose.

20. The production system of claim 19, wherein the stabilized adenosine triphosphate regenerating enzyme comprises a kinase.

21. The production system of claim 19, wherein the stabilized adenosine triphosphate regenerating enzyme comprises a polyphosphate kinase.

22. The production system of any one of claims 19 to 21, wherein the electron donating source comprises nicotinamide adenine dinucleotide phosphate or a reduced form of nicotinamide adenine dinucleotide phosphate, or any combination thereof.

23. The production system of claim 22, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

24. The production system of any one of claims 19 to 21, wherein the electron donating source is an electrode.

25. The production system of any one of claims 19 to 24, wherein the phosphate agent comprises polyphosphate.

26. A production system for producing glucose from carbon dioxide, comprising:
a carbon dioxide source configured to output carbon dioxide;
a ribulose 1,5-bisphosphate configured to output ribulose 1,5-bisphosphate;
a phosphate source configured to output a phosphate agent;
a water source configured to output water; and
a reactor configured to:
receive carbon dioxide from the carbon dioxide source, the phosphate agent from the phosphate source, and water from the water source into the reactor containing stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media to:
(i) produce glyceraldehyde 3-phosphate,
(ii) regenerate adenosine triphosphate, and
(iii) convert glyceraldehyde 3-phosphate to glucose.

27. The production system of claim 26, wherein the stabilized adenosine triphosphate regenerating enzyme comprises a kinase.

28. The production system of claim 26, wherein the stabilized adenosine triphosphate regenerating enzyme comprises a polyphosphate kinase.

29. The production system of any one of claims 26 to 28, wherein the electron donating source comprises nicotinamide adenine dinucleotide phosphate or a reduced form of nicotinamide adenine dinucleotide phosphate, or any combination thereof.

30. The production system of claim 29, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

31. The production system of any one of claims 26 to 28, wherein the electron donating source is an electrode.

32. The production system of any one of claims 26 to 31, wherein the phosphate agent comprises polyphosphate.

33. A production system for producing glucose from carbon dioxide, comprising:
a carbon dioxide source configured to output carbon dioxide;
an adenosine triphosphate source configured to output adenosine triphosphate;
a water source configured to output water; and
a reactor configured to:
receive carbon dioxide from the carbon dioxide source, adenosine triphosphate from the adenosine triphosphate source, and water from the water source into the reactor containing ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media to:
(i) produce glyceraldehyde 3-phosphate,
(ii) regenerate ribulose 1,5-bisphosphate, and
(iii) convert glyceraldehyde 3-phosphate to glucose.

34. The production system of claim 33, wherein the electron donating source comprises nicotinamide adenine dinucleotide phosphate or a reduced form of nicotinamide adenine dinucleotide phosphate, or any combination thereof.

35. The production system of claim 34, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

36. The production system of any one of claims 33 to 35, wherein the electron donating source is an electrode.

37. A production system for producing glucose from glyceraldehyde 3-phosphate, comprising:
a glyceraldehyde 3-phosphate source configured to output glyceraldehyde 3-phosphate;
a water source configured to output water; and
a reactor configured to:
receive glyceraldehyde 3-phosphate from the glyceraldehyde 3-phosphate source and water from the water source into the reactor containing, stabilized aldolase, stabilized fructose 1,6-bisphosphatase, stabilized phosphoglucose isomerase, and stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase in an aqueous media to produce glucose.

38. A production system for producing cellulose from carbon dioxide, comprising:
a carbon dioxide source configured to output carbon dioxide;
a phosphate source configured to output a phosphate agent;
a water source configured to output water; and
a reactor configured to:
receive carbon dioxide from the carbon dioxide source, the phosphate agent from the phosphate source, and water from the water source into the reactor containing ribulose-1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media to:
(i) produce glyceraldehyde 3-phosphate,
(ii) regenerate ribulose 1,5-bisphosphate,
(iii) regenerate adenosine triphosphate,
(iv) convert glyceraldehyde 3-phosphate to glucose,
(v) convert glucose to cellulose, and
(vi) regenerate uridine triphosphate.

39. The production system of claim 38, wherein the stabilized adenosine triphosphate regenerating enzyme comprises a kinase.

40. The production system of claim 38, wherein the stabilized adenosine triphosphate regenerating enzyme comprises a polyphosphate kinase.

41. The production system of claim 38, wherein the stabilized uridine triphosphate regenerating enzyme comprises a stabilized kinase.

42. The production system of claim 38, wherein the stabilized uridine triphosphate regenerating enzyme comprises a polyphosphate kinase.

43. The production system of any one of claims 38 to 42, wherein the electron donating source comprises nicotinamide adenine dinucleotide phosphate or a reduced form of nicotinamide adenine dinucleotide phosphate, or any combination thereof.

44. The production system of claim 43, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

45. The production system of any one of claims 38 to 42, wherein the electron donating source is an electrode.

46. The production system of any one of claims 38 to 45, wherein the phosphate agent comprises polyphosphate.

47. A production system for producing cellulose from carbon dioxide, comprising:
a carbon dioxide source configured to output carbon dioxide;
a ribulose 1,5-bisphosphate configured to output ribulose 1,5-bisphosphate;
a phosphate source configured to output a phosphate agent;
a water source configured to output water; and
a reactor configured to:
receive carbon dioxide from the carbon dioxide source, ribulose 1,5-bisphosphate from the ribulose 1,5-bisphosphate source, the phosphate agent from the phosphate source, and water from the water source into the reactor containing stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media to:
(i) produce glyceraldehyde 3-phosphate,
(ii) regenerate adenosine triphosphate,
(iii) convert glyceraldehyde 3-phosphate to glucose,
(iv) convert glucose to cellulose, and
(v) regenerate uridine triphosphate.

48. The production system of claim 47, wherein the stabilized adenosine triphosphate regenerating enzyme comprises a kinase.

49. The production system of claim 47, wherein the stabilized adenosine triphosphate regenerating enzyme comprises a polyphosphate kinase.

50. The production system of claim 47, wherein the stabilized uridine triphosphate regenerating enzyme comprises a stabilized kinase.

51. The production system of claim 47, wherein the stabilized uridine triphosphate regenerating enzyme comprises a polyphosphate kinase.

52. The production system of any one of claims 47 to 51, wherein the electron donating source comprises nicotinamide adenine dinucleotide phosphate or a reduced form of nicotinamide adenine dinucleotide phosphate, or any combination thereof.

53. The production system of claim 52, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

54. The production system of any one of claims 47 to 51, wherein the electron donating source is an electrode.

55. The production system of any one of claims 47 to 54, wherein the phosphate agent comprises polyphosphate.

56. A production system for producing cellulose from carbon, comprising:
a carbon dioxide source configured to output carbon dioxide;
a phosphate source configured to output a phosphate agent;
an adenosine triphosphate source configured to output adenosine triphosphate;
a water source configured to output water; and
a reactor configured to:
receive carbon dioxide from the carbon dioxide source, the phosphate agent from the phosphate source, adenosine triphosphate from the adenosine triphosphate source, and water from the water source into the reactor containing ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media to:
(i) produce glyceraldehyde 3-phosphate,
(ii) regenerate ribulose 1,5-bisphosphate,
(iii) convert glyceraldehyde 3-phosphate to glucose,
(iv) convert glucose to cellulose, and
(v) regenerate uridine triphosphate.

57. The production system of claim 56, wherein the stabilized uridine triphosphate regenerating enzyme comprises a stabilized kinase.

58. The production system of claim 56, wherein the stabilized uridine triphosphate regenerating enzyme comprises a polyphosphate kinase.

59. The production system of any one of claims 56 to 58, wherein the electron donating source comprises nicotinamide adenine dinucleotide phosphate or a reduced form of nicotinamide adenine dinucleotide phosphate, or any combination thereof.

60. The production system of claim 59, wherein the reactor further contains a stabilized glucose dehydrogenase enzyme for regenerating nicotinamide adenine dinucleotide phosphate or the reduced form of nicotinamide adenine dinucleotide phosphate, or any combination thereof.

61. The production system of any one of claims 56 to 58, wherein the electron donating source is an electrode.

62. The production system of any one of claims 56 to 61, wherein the phosphate agent comprises polyphosphate.

63. A production system for producing cellulose from glyceraldehyde 3-phosphate, comprising:
a glyceraldehyde 3-phosphate source configured to output glyceraldehyde 3-phosphate;
a phosphate source configured to output a phosphate agent;
a water source configured to output water; and
a reactor configured to:
receive glyceraldehyde 3-phosphate from the glyceraldehyde 3-phosphate source, the phosphate agent from the phosphate source, and water from the water source into the reactor containing, adenosine triphosphate, a stabilized adenosine triphosphate regenerating enzyme, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, stabilized phosphoglucose isomerase, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media to:
(i) convert glyceraldehyde 3-phosphate to glucose,
(ii) regenerate adenosine triphosphate,
(iii) convert glucose to cellulose, and
(iv) regenerate uridine triphosphate.

64. The production system of claim 63, wherein the stabilized adenosine triphosphate regenerating enzyme comprises a kinase.

65. The production system of claim 63, wherein the stabilized adenosine triphosphate regenerating enzyme comprises a polyphosphate kinase.

66. The production system of claim 63, wherein the stabilized uridine triphosphate regenerating enzyme comprises a stabilized kinase.

67. The production system of claim 63, wherein the stabilized uridine triphosphate regenerating enzyme comprises a polyphosphate kinase.

68. The production system of any one of claims 63 to 67, wherein the phosphate agent comprises polyphosphate.

69. A production system for producing cellulose from glucose, comprising:
a glucose source configured to output glucose;
a phosphate source configured to output a phosphate agent; and
a reactor configured to:
receive glucose from the glucose source and the phosphate agent from the phosphate source into the reactor containing adenosine triphosphate, a stabilized adenosine triphosphate regenerating enzyme, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media to:
(i) convert glucose to cellulose,
(ii) regenerate adenosine triphosphate, and
(iii) regenerate uridine triphosphate.

70. The production system of claim 69, wherein the stabilized adenosine triphosphate regenerating enzyme comprises a kinase.

71. The production system of claim 69, wherein the stabilized adenosine triphosphate regenerating enzyme comprises a polyphosphate kinase.

72. The production system of claim 69, wherein the stabilized uridine triphosphate regenerating enzyme comprises a stabilized kinase.

73. The production system of claim 69, wherein the stabilized uridine triphosphate regenerating enzyme comprises a polyphosphate kinase.

74. The production system of any one of claims 60 to 73, wherein the phosphate agent comprises polyphosphate.

75. A method for producing glyceraldehyde 3-phosphate from carbon dioxide, comprising:
(i) combining carbon dioxide, a phosphate agent, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media;
(ii) producing glyceraldehyde 3-phosphate; and
(iii) regenerating ribulose 1,5-bisphosphate and adenosine triphosphate.

76. A method for producing glyceraldehyde 3-phosphate from carbon dioxide, comprising:
(i) combining carbon dioxide, a phosphate agent, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, and an electron donating source in an aqueous media;
(ii) producing glyceraldehyde 3-phosphate, and
(ii) regenerating adenosine triphosphate.

77. A method for producing glyceraldehyde 3-phosphate from carbon dioxide, comprising:
(i) combining carbon dioxide, adenosine triphosphate, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, and an electron donating source in an aqueous media;
(ii) producing glyceraldehyde 3-phosphate; and
(iii) regenerating ribulose 1,5-bisphosphate.

78. A method for producing glucose from carbon dioxide, comprising:
(i) combining carbon dioxide, a phosphate agent, water, ribulose 1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating agent in an aqueous media;
(ii) producing glyceraldehyde 3-phosphate;
(iii) regenerating ribulose 1,5-bisphosphate and adenosine triphosphate; and
(iv) converting glyceraldehyde 3-phosphate to glucose.

79. A method for producing glucose from carbon dioxide, comprising:
(i) combining carbon dioxide, ribulose 1,5-bisphosphate, a phosphate agent, water, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media;
(ii) producing glyceraldehyde 3-phosphate;
(iii) regenerating adenosine triphosphate; and
(iv) converting glyceraldehyde 3-phosphate to glucose.

80. A method for producing glucose from carbon dioxide, comprising:
(i) combining carbon dioxide, adenosine triphosphate, water, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, and an electron donating source in an aqueous media;
(ii) producing glyceraldehyde 3-phosphate;
(ii) regenerating ribulose 1,5-bisphosphate; and
(iv) converting glyceraldehyde 3-phosphate to glucose.

81. A method for producing glucose from glyceraldehyde 3-phosphate, comprising:
(i) combining glyceraldehyde 3-phosphate, water, stabilized aldolase, stabilized fructose 1,6-bisphosphatase, stabilized phosphoglucose isomerase, and stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase in an aqueous media; and
(ii) producing glucose.

82. A method for producing cellulose from carbon dioxide, comprising:
(i) combining carbon dioxide, a phosphate agent, water, ribulose-1,5-bisphosphate, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media;
(ii) producing glyceraldehyde 3-phosphate;
(iii) regenerating ribulose 1,5-bisphosphate and adenosine triphosphate, and uridine triphosphate; and
(iv) converting glyceraldehyde 3-phosphate to glucose and glucose to cellulose. and

83. A method for producing cellulose from carbon dioxide, comprising:
(i) combining carbon dioxide, ribulose 1,5-bisphosphate, a phosphate agent, water, stabilized ribulose-1,5-bisphosphate carboxylase-oxygenase, adenosine triphosphate, a stabilized adenosine triphosphate regenerating enzyme, stabilized phosphoglycerate kinase, stabilized glyceraldehyde 3-phosphate dehydrogenase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media;
(ii) producing glyceraldehyde 3-phosphate;
(iii) regenerating adenosine triphosphate and uridine triphosphate; and
(iv) converting glyceraldehyde 3-phosphate to glucose and glucose to cellulose.

84. A method for producing cellulose from carbon dioxide, comprising:
(i) combining carbon dioxide, a phosphate reagent, water, ribulose 1,5-bisphosphate, stabilized ribulose-1,5-bisphosphate carboxylase-oxygenase, stabilized phosphoglycerate kinase, stabilized glyceraldehyde 3-phosphate dehydrogenase, stabilized transketolase, stabilized ribulose-5-phosphate kinase, stabilized aldolase, stabilized triosephosphate isomerase, stabilized fructose 1,6-bisphosphatase, stabilized phosphopentose epimerase, stabilized ribose-5-phosphate isomerase, stabilized sedoheptulose 1,7-bisphosphatase, stabilized phosphoribulokinase, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, uridine triphosphate, a stabilized uridine triphosphate regenerating enzyme, stabilized glucokinase, stabilized phosphoglucomutase, stabilized glucose-1-phosphate uridylyltransferase, stabilized cellulose synthase, and an electron donating source in an aqueous media;
(ii) producing glyceraldehyde 3-phosphate;
(iii) regenerating ribulose 1,5-bisphosphate and uridine triphosphate; and
(iv) converting glyceraldehyde 3-phosphate to glucose and glucose to cellulose.

85. A method for producing cellulose from glyceraldehyde 3-phosphate, comprising:
(i) combining glyceraldehyde 3-phosphate, a phosphate agent, water, adenosine triphosphate, a stabilized adenosine triphosphate regenerating enzyme, stabilized aldolase, stabilized fructose 1,6-bisphosphatase, stabilized phosphoglucose isomerase, stabilized glucose 6-phosphatase, stabilized triosephosphase isomerase, stabilized glyceraldehyde stabilized phosphate dehydrogenase, stabilized phosphoglycerate kinase, stabilized phosphoglycerate mutase, stabilized enolase, stabilized phosphoenolpyruvate carboxykinase, stabilized pyruvate carboxylase, stabilized phosphoglucose isomerase, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media;
(ii) converting glyceraldehyde 3-phosphate to glucose;
(iii) regenerating adenosine triphosphate and uridine triphosphate; and
(iv) converting glucose to cellulose.

86. A method for producing cellulose from glucose, comprising:
(i) combining glucose, a phosphate agent, adenosine triphosphate, a stabilized adenosine triphosphate regenerating enzyme, uridine triphosphate, a stabilized uridine triphosphate regenerating enzyme, stabilized glucokinase, stabilized phosphoglucomutase, stabilized glucose-1-phosphate uridylyltransferase, and stabilized cellulose synthase in an aqueous media;
(ii) converting glucose to cellulose; and
(iii) regenerating adenosine triphosphate and uridine triphosphate.
